(19) United States
(12) Patent Application Publication
(10) Pub. No.: US 2017/0283859 A1
(43) Pub. Date: Oct. 5, 2017

(54) MICROFLUIDIC qRT-PCR ANALYSIS OF SINGLE CELLS

(71) Applicant: The Trustees Of Columbia University In the City of New York, New York, NY (US)

(72) Inventors: Qiao Lin, New York, NY (US); Jing Zhu, Brisbane, CA (US); Timothy R. Olsen, New York, NY (US); Hao Sun, Fuzhou (CN); Brian Ponnaiya, Ossinin, NY (US); Sally A. Amundson, New York, NY (US); David J. Brenner, New York, NY (US)

(73) Assignee: The Trustees Of Columbia University In the City of New York, New York, NY (US)

(21) Appl. No.: 15/492,656

(22) Filed: Apr. 20, 2017

Related U.S. Application Data
(63) Continuation of application No. PCT/US2015/057086, filed on Oct. 23, 2015.
(60) Provisional application No. 62/068,527, filed on Oct. 24, 2014.
(60) Provisional application No. 62/068,432, filed on Oct. 24, 2014.
(60) Provisional application No. 62/069,122, filed on Oct. 27, 2014.
(60) Provisional application No. 62/069,117, filed on Oct. 27, 2014.
(60) Provisional application No. 62/133,230, filed on Mar. 13, 2015.
(60) Provisional application No. 62/133,227, filed on Mar. 13, 2015.

(51) Int. Cl. C12Q 1/68; B01L 7/00; B01L 3/00

(52) U.S. Cl. CPC C12Q 1/686; B01L 3/502761; B01L 3/502753; B01L 3/502738; B01L 7/52; C12Q 1/6806; B01L 2200/10; B01L 2200/0647; B01L 2300/123; B01L 2300/1827; B01L 2300/0627; B01L 2300/02; B01L 2400/0655; B01L 2200/142

(57) ABSTRACT

The disclosed subject matter provides a microdevice and techniques for single-cell gene expression profiling using a microfluidic device capable of cell-trapping, cell lysis, bead-based gene analysis. The microdevice can be capable of independent or parallelized, simultaneous quantitative genetic assays of single cells.

### CROSS REFERENCE TO RELATED APPLICATIONS

This application is a continuation of International Patent Application No. PCT/US2015/057086, filed Oct. 23, 2015, which claims priority from U.S. Provisional Application No. 62/068,432, filed Oct. 24, 2014; U.S. Provisional Application No. 62/068,527, filed Oct. 24, 2014; U.S. Provisional Application No. 62/069,117, filed Oct. 27, 2014; U.S. Provisional Application No. 62/069,122, filed Oct. 27, 2014; U.S. Provisional Application No. 62/133,227, filed Mar. 13, 2015; and U.S. Provisional Application No. 62/133,230, filed Mar. 13, 2015, each of which is incorporated by reference herein in their entirety.

### STATEMENT REGARDING FEDERALLY FUNDED RESEARCH

This invention was made with government support under 5U19AI067773 and 8R21GM104204 awarded by the National Institutes of Health. The government has certain rights in this invention.

### BACKGROUND

A challenge in gene expression profiling is the ubiquitous cellular heterogeneity existing in biological samples. Conventionally, gene expression assays can be focused on groups of cells from organs, tissues, or cell culture as the measurement technologies have been limited by accuracy, sensitivity, and dynamic range. While cells can appear morphologically identical, recent evidence reveals that the gene expression level of individual cells in a population can vary due to cellular heterogeneity. Thus, gene expression studies using groups of cells can fail to detect differences in the molecular composition of individual cells.

Single-cell gene expression profiling, a method to assay the gene patterns in individual cells, can alleviate the complexity of genetic variability caused by heterogeneity and has the potential to reveal intracellular molecular mechanisms and pathways. By quantifying the alterations in gene expression, the influence of stimuli on genes can be identified. Certain methods combine next-generation nucleic acid sequencing with improved biochemical methodologies such as template switching technology (Smart-seq), transcriptome in vivo analysis (TIVA), unique molecular identifiers (UMis) and fluorescent in situ RNA sequencing (FISSEQ), and genetic analysis at the single cell or single molecule level has been used in applications such as personalized therapy, drug discovery, and embryonic stem cell research. Such assays, however, have been technically challenging due to the low quantity and degradation of RNA from an individual cell.

Microfluidic technology can provide rapid, sensitive, and quantitative assays in small sample volumes while eliminating the need for labor intensive and potentially error-prone laboratory manipulation. Certain devices, however, use solution-based methods, which do not allow efficient manipulation of samples and reagents or retrieval of reaction products for real-time analysis of singe-cell gene expression profiling. Certain microchip approaches require off-chip manual transfer of RNA (which is a common source of potential contamination to the samples), relies on off-chip thermal control instrumentation, and/or involves rather complicated flow control components and operations.

There is a need to develop a microfluidic device that integrates isolation, immobilization and lysis of single cells as well as purification, reverse transcription (RT) and quantitative real-time PCR (qPCR) of messenger RNA (mRNA), without requiring off-chip manual transfer of cells and reagents between the individual reaction steps, and without using off-chip qPCR instruments. There also exists the need for a microfluidic device that can perform single-cell gene expression analysis in a single unit while at the same time assaying multiple samples in a parallel fashion (i.e., more than one single cell at a time) to improve throughput.

### SUMMARY

The disclosed subject matter provides a microdevice and techniques for single-cell gene expression profiling. In one aspect, the disclosed subject matter provides a microdevice and techniques for single-cell gene expression profiling using a microfluidic chip capable of cell-trapping, cell lysis, and bead-based RT-qPCR. In certain embodiments, hydrodynamic forces can be employed for efficient and reliable isolation and immobilization of single cells, for downstream quantitative single-cell genetic analysis including cell lysis, mRNA purification, reverse transcription and DNA duplication. In certain embodiments, the microdevice includes polydimethylsiloxane.

In one aspect, a microdevice includes a temperature control chip with an integrated heater and temperature sensor, a reaction chamber, and a cell trapping unit. In certain embodiments, the microdevice includes a cell inlet configured to receive a fluid containing a plurality of cells and one or more analysis units coupled to the cell inlet, where each of the analysis units can include a cell trap configured to trap a single cell from the plurality of cells. In certain embodiments, the cell trapping unit can be equipped with a cell trapping outlet, a cell washing outlet, and/or microvalves.

In one aspect, a microfluidic device can include a cell trap including a flow constriction formed by a narrowing in a microchannel between a first microvalve and a second microvalve. In certain embodiments, the narrowing of the microstructure can reduce the size of the microchannel to less than the average diameter of the cell to be trapped. In certain embodiments, the cell trapping outlet can allow a fluid to flow through the trap and through a cell washing outlet for purging the device of excess cells once a cell is trapped. In certain embodiments, once immobilized, the single cell can be lysed chemically and mRNA templates from the lysate can be captured using microbeads. In certain embodiments, the microbeads can be magnetic. In certain embodiments, the microbeads can include primers configured to capture mRNA. In certain embodiments, the microbeads carrying the mRNA travel to the reaction chamber to initiate RT-qPCR.

In one aspect, reaction chamber can be coupled to the cell trap, and magnetic microbeads including a primer configured to capture mRNA obtained by lysing the single cell can transport the mRNA from the cell trap to the reaction chamber. In certain embodiments, external magnets are used to transport and hold the microbeads in place. In certain embodiments, microvalves can be used to direct and prevent the flow of fluid within the microdevice. Gene expression analysis of the captured mRNA can include RT and/or qPCR.

In one aspect, a microdevice includes a single analysis unit. The single analysis unit microdevice provides a platform for gene detection and sequencing of a single cell. In certain embodiments, a single analysis unit microdevice includes a single analysis unit connected to a main inlet and main outlet, based on a single substrate with an integrated micro heater and temperature sensor. In certain embodiments, the single analysis unit microdevice can include of a cell trap, a cell inlet, a buffer outlet, and a cell outlet; a microbead/gene analysis reagent inlet; and a reaction chamber microchamber.

In one aspect, a microdevice includes two or more analysis units (i.e., an array), which can be capable of parallelized, simultaneous quantitative genetic assays of single cells, thereby providing a platform for multiplex gene detection and sequencing and allowing studies of heterogeneity in biological systems at the single-cell level. In certain embodiments, a microdevice can include of at least two parallel analysis units connected to a single main inlet and main outlet, based on a single substrate with an integrated micro heater and temperature sensor. In certain embodiments, the analysis units can be identical in design and each includes a cell trap, a buffer outlet, a cell outlet and a reaction chamber microchamber. In certain embodiments, an integrated resistive heater and temperature sensor can allow all of the reaction chambers to be thermal cycled individually and simultaneously. In certain embodiments, individual microvalves can be arranged in a combinatorial array to allow precise control of flow within a single analysis unit while mutually preventing flow in the other analysis units. In certain embodiments, a microdevice array can include from two to six or more analysis units.

In one aspect, the disclosed subject matter provides methods for immobilizing, lysing, and performing transcriptional profiling analysis of a single cell. In certain embodiments, the methods can be applied to gene regulation studies of healthy and/or diseased cells. In certain embodiments, the methods can be applied to gene regulation studies by treating cells with a drug to detect drug induced single cell gene expression level alterations.

The sample can be derived from a bodily fluid, a tissue sample, or cell culture. In certain embodiments, the bodily fluid, tissue sample, or cells can be obtained from a human or animal.

### BRIEF DESCRIPTIONS OF THE DRAWINGS

FIGS. 1A-1B: A) Design of a single analysis unit microdevice according to some embodiments of the disclosed subject matter; B) Bead-based RT-qPCR principle according to some embodiments of the disclosed subject matter.

FIGS. 2A-2B: A) Design of a microdevice array according to some embodiments of the disclosed subject matter. B) Top and cross section views of a single analysis unit of the array, with a trapped single cell shown in the inset.

FIGS. 3A-3D: Fabrication process of the microfluidic device according to some embodiments of the disclosed subject matter.

FIG. 4: A flowchart illustrating an exemplary embodiment of a method for isolating, lysing, and analyzing gene expression of a single cell.

FIG. 5: Illustrates certain elements of an analysis unit of a microdevice according to some embodiments of the disclosed subject matter.

FIGS. 6A-6B: Schematic of an example set-up according to some embodiments of the disclosed subject matter. A) Is a schematic for a single analysis unit microdevice with a photograph insert of the packaged device. B) Is a schematic for a microdevice array with a photograph insert of a single microvalve (e.g., pressurized microvalve).

FIGS. 7A-7H: Demonstration of the on-chip flow control according to some embodiments of the disclosed subject matter.

FIG. 8: A plot showing the resistance of temperature sensor (R) and its dependence on temperature (T).

FIG. 9: A plot showing time-resolved tracking of the chamber temperature.

FIG. 10: A plot showing validation of on-chip RT-PCR.

FIG. 11: A plot showing microbead quantity analysis. 3.75×10⁶ oligo(dT)₂₅ beads trapped 10⁵ XenoRNA copies efficiently.

FIG. 12: A plot showing quantified detection of mRNA trapping efficiency using 3.75×10⁶ beads.

FIG. 13: A plot showing the mean and standard deviation of on-chip and in-tube RT-qPCR (with the Cq value at the 10 000 XenoRNA copy number determined to be beyond the imaging system's measurement range and hence omitted from the linear fit).

FIGS. 14A-14D: Testing of single-cell trapping and lysis. A) Micrographs of single-cell trapping. B) Flow rate effect on cell trapping time and probability. Trapping time and probability decrease with increased flow rates. C) Single-cell trapping efficiency. Maximum trapping efficiency obtained at a flow rate of 15 nl s⁻¹ and a cell density of 10⁵ cells per ml. D) Lysis efficiency.

FIGS. 15A-15B: Fully integrated on-chip single-cell RT-qPCR according to some embodiments of the disclosed subject matter. A) Amplification curves of MMS treated (thick black line) and untreated (thin gray line) single-cell RT-qPCR. B) Mean Cq values for integrated RT-qPCR in treated and untreated single cells were obtained from five repeated tests.

FIG. 16: A plot showing the steady ROX fluorescent intensity, in addition to the constant pathlength during the qPCR process indicates stable reagent concentrations.

FIG. 17: A plot showing now template control (NTC) testing of the fully integrated on-chip single-cell RT-qPCR.

FIG. 18: A microdevice array according to some embodiments of the disclosed subject matter. For visualization, the fluid paths and control channels were loaded with dyes.

FIGS. 19A-19D: Fabrication process of the microchip array according to some embodiments of the disclosed subject matter. (A) Heater and sensor with passivation layer. (B) Control layer and featureless PDMS membrane. (C) Flow layer bonding with control layer (D) Device package using oxygen plasma.

FIG. 20: A plot showing on-chip temperature control.

FIG. 21: A plot showing on-chip RNA capture capacity testing with varying XenoRNA copy numbers, no-template control (NTC) and positive control (PC).

FIGS. 22A-22B: A plot showing A) parallelized RT-qPCR pf XenoRNA templates showing consistency in gene expression analysis in different analysis units of the array and B) single-cell gene expression with ROX and FAM signals around the quantification cycles of a sample with 1×10⁵ copies of XenoRNA.

FIG. 23: A plot showing validation of on-chip RT-PCR using synthetically homogenized XenoRNA templates and no-template control (NTC).

FIG. 24: A plot showing validation and consistency of on-chip RT-qPCR testing using parallelized single-cell RT-qPCR of XenoRNA templates and no-template control (NTC).

FIG. 25: A plow showing fitting curves of on-chip and in-tube RT-qPCR for XenoRNA templates of different copy number.

FIG. 26: A plot showing the endpoint fluorescent intensity of on-chip tests after 40-cycle qPCR. Data points represent ten repeated tests.

FIG. 27: A plot showing endpoint fluorescent intensity of in-tube tests after 40-cycle qPCR.

FIG. 28: A plow showing mean fluorescent intensity of end-point RT-qPCR of MCF-7 cells and no-template control (NTC). Three samples were used for each and error bars represent standard.

FIG. 29: A plow showing on-chip single-cell RT-qPCR measuring the induction of the GAPDH and CDKN1A.

FIGS. 30A-30D: (A) The amplification curves of CDKNIA and GAPDH in MMS treated, untreated single MCF-7 cells and no-template control (NTC) by the microfluidic array. (B) qPCR Cq values of CDKNIA and GAPDH in MMS treated and untreated single MCF-7 cells. (C) qPCR Cq values with amplification curves shown in the inset of CDKN1A in single cells exposed to MMS for different time durations. (D) qPCR Cq values with amplification curves shown in the inset of CDKN1A in single cells treated with different doses of MMS.

### DETAILED DESCRIPTION

The disclosed subject matter provides for devices and methods for single-cell gene expression profiling. More specifically, the disclosed subject matter provides for a microfluidic device capable of cell-trapping, cell lysis, bead-based RT-qPCR, and uses thereof.

As used herein, the term “analysis unit” includes a cell trap, a reaction chamber, and microbeads for capturing the cellular mRNA.

As used herein, the term “array” means one or more analysis units. For example, an array can be made up of at least two, at least three, at least four, at least five, or at least six analysis or more units. In certain embodiments, the array can be made up for hundreds or thousands of units.

As used herein, the term “cell suspension” refers to a plurality of cells that have been dissociated in a buffer resulting in single cells being suspended in the buffer. The cell suspension can have a certain cell count per volume. By way of example, but not limited to, the cell suspension can have 10⁵ cell per mL of buffer or solution.

As used herein, the term “RT-qPCR” refers to quantitative reverse transcription PCR. RT-qPCR is a method in which RNA is first transcribed into complementary DNA (“cDNA”) by reverse transcriptase from total messenger RNA (“mRNA”). The cDNA is then used as the template for the qPCR reaction for gene expression analysis.

As used herein, the term “functionalized” means to introduce functional groups to the surface. The functional groups can be covalently attached or grafted to the surface of the functionalized substrate. For example, the functional groups can include material that can capture genetic material (e.g., primers).

### The Microfluidic Device

The disclosed subject matter provides a microfluidic devise capable of cell-trapping, cell lysis, and bead-based RT-qPCR on a single cell. In certain embodiments, hydrodynamic forces can be employed for efficient and reliable isolation and immobilization of single cells, for downstream quantitative single-cell genetic analysis including cell lysis, mRNA purification, reverse transcription, and DNA duplication.

Once immobilized, single cells can be lysed chemically and mRNA templates from the lysate can be captured using microbeads. In certain embodiments, the microbeads can specifically target and capture mRNA molecules from virtually any crude sample and eliminate the need to purify total RNA when the desired information-bearing nucleic acid is mRNA. In certain embodiments, the microbeads carrying the mRNA travel to the reaction chamber to initiate RT-qPCR.

In certain embodiments, the microfluidic device can include a single analysis unit. In certain embodiments, the microfluidic device can include multiple analysis units (i.e., an array). In certain embodiments, the microfluidic device can in include 1 to 10,000 units. In certain embodiments, the microfluidic device can in include 1 to 10 units, 2 to 10 units, 3 to 9 units, 4 to 8 units, or 5 to 7 units. In certain embodiments, the microfluidic device can include at least 1, at least 2, at least 3, at least 4, at least 5, or at least 6 analysis units, at least 7, at least 8, at least 9, or at least 10 or more units. In certain embodiments, the microfluidic device can include 10 to 100 units. In certain embodiments, the microfluidic device can in include 20 to 100 units, 30 to 90 units, 40 to 80 units, or 50 to 70 units. In certain embodiments, the microfluidic device can include 100 to 1000 units. In certain embodiments, the microfluidic device can in include 200 to 1000 units, 300 to 900 units, 400 to 800 units, or 500 to 700 units. In certain embodiments, the microfluidic device can include 1000 to 10,000 units. In certain embodiments, the microfluidic device can in include 2000 to 10,000 units, 3000 to 9000 units, 4000 to 8000 units, or 5000 to 7000 units.

### Single Analysis Unit

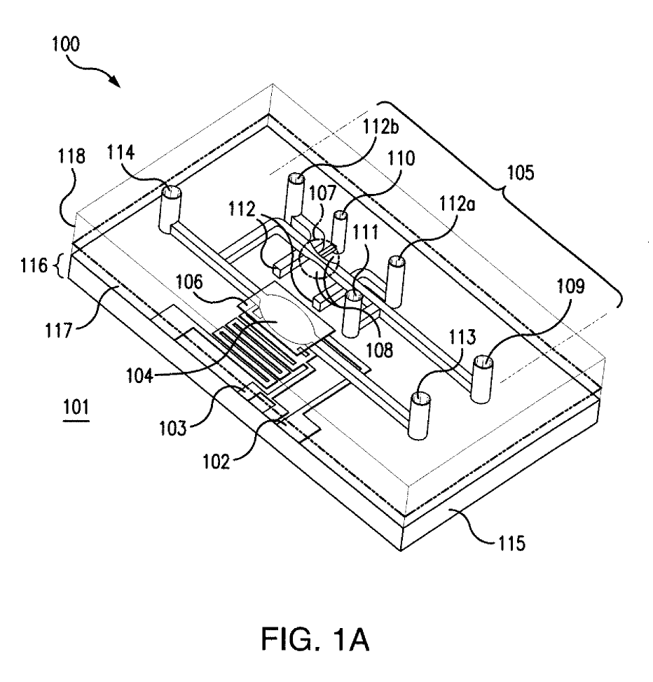

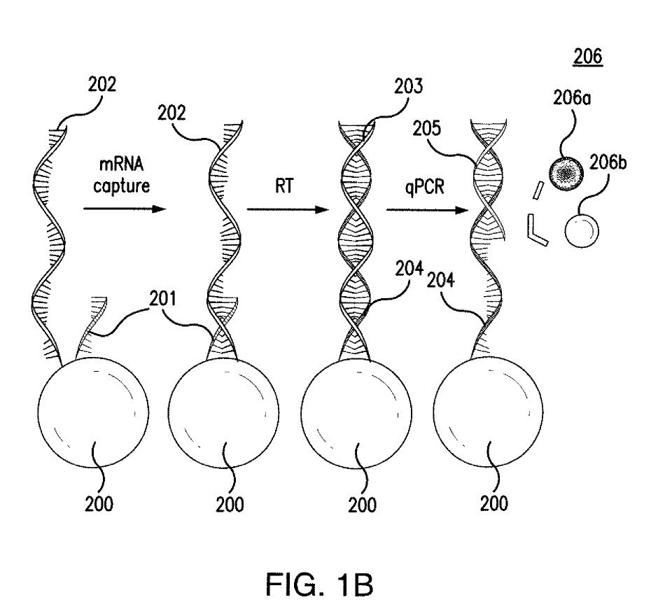

An exemplary embodiment of a microfluidic device (100) with a single analysis unit (101) in accordance with the disclosed subject matter is illustrated in FIG. 1A. As shown in FIG. 1A, the microdevice (100) can include a temperature control chip with an integrated heater (102) and temperature sensor (103), a reaction chamber (104), and a cell trapping unit (105). The cell trapping unit consists of the valves (112b) and (112a), the cell trap (107), the cell trapping outlet (110), the cell washing outlet (111), and the cell inlet (109).

The heater (102) and temperature sensor (103) can be integrated beneath the center of the reaction chamber (104). In certain embodiments, the heater (102) and temperature sensor (103) can be integrated between a substrate (115) and the control layer (117). In certain embodiments, the heater (102) and temperature sensor (103) can be integrated between a substrate (115) and the flow layer (118). Together the temperature sensor (103), heater (102) (e.g., electrodes), and substrate (115) make up the electrode/substrate layer (116).

In certain embodiments, the heater (102) and temperature sensor (103) can be made of chrome (e.g., 10 nm) and gold (e.g., 100 nm) thin films. In certain embodiments, the electrodes can be made of chrome, gold, platinum, aluminum, titanium, or combinations thereof. In certain embodiments, the substrate (115) can be made of a transparent material. The substrate (115) can be made, but not limited to, glass (e.g., a glass slide), clear polymers (e.g., vinyl, acrylic, Plexiglass). In certain embodiments, the substrate (115) is impermeable to water and other liquids.

In certain embodiments, the heater (102) and temperature sensor (103) can be serpentine-shaped. The heater can be, for example, a resistive heater. The temperature sensor (103) can be configured to measure a temperature of the reaction chamber (104). In certain embodiments, the temperature sensor (103) has a linewidth of about 20 μm to about 100 μm. In certain embodiments, the temperature sensor (103) has a linewidth of about 50 μm. In certain embodiments, the temperature sensor (103) has a linewidth of about 25 μm to about 95 μm, about 30 μm to about 90 μm, about 35 μm to about 85 μm, about 40 μm to about 80 μm, about 45 μm to about 75 μm, about 50 μm to about 70 μm, or about 55 μm to about 65 μm. In certain embodiments, the heater (102) has a linewidth of about 200 μm to about 1000 μm. In certain embodiments, the heater (102) has a linewidth of about 400 μm. In certain embodiments, the heater (102) has a linewidth of about 250 μm to about 950 μm, about 300 μm to about 900 μm, about 350 μm to about 850 μm, about 400 μm to about 800 μm, about 450 μm to about 750 μm, about 500 μm to about 700 μm, or about 550 μm to about 650 μm.

The reaction chamber (104) is in the flow layer (118) of the microfluidic device. In certain embodiments, the reaction chamber (104) is designed for a two-step RT-qPCR process or a three-step RT-qPCR process. In certain embodiments, the reaction chamber (104) can be made of polydimethylsiloxane (PDMS). The reaction chamber (104) can be elliptical, cylindrical, or rectangular with a trapezoid on the top and bottom. In certain embodiments, the reaction chamber (104) can be any shape with a flat bottom. In certain embodiments, the reaction chamber (104) can be elliptical with the dimensions of about 5 mm to about 10 mm in length, about 2 mm to about 6 mm in width, and about 10 μm to about 25 μm in height. In certain embodiments, the reaction chamber (104) is elliptical with the dimensions of about 7.7 mm in length, about 5.7 mm in width, and about 15 μm in height. In certain embodiments, the reaction chamber (104) can have the dimensions of about 5 mm to about 10 mm in length, about 6 mm to about 9 mm in length, or about 7 mm to about 8 mm in length. In certain embodiments, the reaction chamber (104) can have the dimensions of about 2 mm to about 6 mm in width, about 2.5 mm to about 5.5 mm in width, about 3 mm to about 5 mm in width, or about 3.5 mm to about 4.5 mm in width. In certain embodiments, the reaction chamber (104) can have the dimensions of about 10 μm to about 25 μm in height, about 12.5 μm to about 22.5 μm in height, or about 15 μm to about 20 μm in height. In certain embodiments, the reaction chamber (104) has a volume of about 100 nL to about 1500 nL. In certain embodiments, the reaction chamber (104) has a volume of about 655 nL. In certain embodiments, the reaction chamber (104) has a volume of about 100 nL to about 1500 nL, about 200 nL to about 1400 nL, about 300 nL to about 1300 nL, about 400 nL to about 1200 nL, about 500 nL to about 1100 nL, about 600 nL to about 1000 nL, or about 700 nL to about 900 nL.

In certain embodiments, the reaction chamber (104) can be covered with an evaporation barrier (106). The inclusion of the evaporation barrier (106) can inhibit reagent evaporation and diffusion caused by porosity of the reaction chamber (e.g. PDMS porosity). In certain embodiments, the evaporation barrier (106) can be transparent. In certain embodiments, the evaporation barrier (106) can include a pressure sensitive adhesive film. The evaporation barrier (106) can be made of, for example but not limited to, polypropylene or polycarbonate. In certain embodiments, the evaporation barrier can be any transparent material. In certain embodiments, the evaporation barrier can be any transparent water impermeable material. In certain embodiments, the evaporation barrier is not autofluorescent. In certain embodiments, the evaporation barrier can be glass. The evaporation barrier (106) can be slightly larger than the reaction chamber (104). For example, the evaporation barrier (106) can have the dimension of about 3 mm in length and 0.5 mm in width. In certain embodiments, the evaporation barrier (106) can have a thickness of about 0.1 to about 1 mm. In certain embodiments, the evaporation barrier (106) can have a thickness of about 0.1 mm. In certain embodiments, the evaporation barrier (106) can have a thickness of about 0.1 mm to about 1 mm, about 0.2 mm to about 0.9 mm, about 0.3 mm to about 0.8 mm, about 0.4 mm to about 0.7 mm, or about 0.5 mm to about 0.6 mm. In certain embodiments, the evaporation barrier (106) is bonded to the top of the reaction chamber (104).

The cell trapping unit (105) is in the flow layer (118) of the microfluidic device. In certain embodiments, the cell trapping unit (105) can have the dimensions of about 500 μm to about 2000 μm in length, about 80 μm to about 120 μm in width, and about 10 μm to about 25 μm in height. In certain embodiments, the cell trapping unit (105) can have the dimensions of about 800 μm in length, about 100 μm in width and about 15 μm in height. In certain embodiments, the cell trapping unit (105) can have the length of about 500 μm to about 2000 μm in length, about 600 μm to about 1900 μm, about 700 μm to about 1800 μm, about 800 μm to about 1700 μm, about 900 μm to about 1600 μm, about 800 μm to about 1500 μm, about 900 μm to about 1400 μm, about 1000 μm to about 1300 μm, or about 1100 μm to about 1200 μm. In certain embodiments, the cell trapping unit (105) can have the width of about 80 μm to about 120 μm, about 85 μm to about 115 μm, about 90 μm to about 110 μm, or about 95 μm to about 105 μm. In certain embodiments, the cell trapping unit (105) can have the height of about 10 μm to about 25 μm, about 11 μm to about 24 μm, about 12 μm to about 23 μm, about 13 μm to about 22 μm, about 14 μm to about 22 μm, about 15 μm to about 21 μm, about 16 μm to about 20 μm, or about 17 μm to about 19 μm.

The cell trapping unit (105) can include a cell trap (107). In certain embodiments, the cell trap (107) can be a neck-shaped channel. The cell trap (107) can be created by a narrowing of the channel (108). In certain embodiments, the narrowing of the chamber is created by the mold for the flow layer. The narrowing of the channel for the cell trap can reduce the channel width to smaller than the average diameter of the cell type to be trapped. In certain embodiments, the channel can be reduced from a width from about 80 μm to about 120 μm down to about 4 μm to about 5 μm. In certain embodiments, the channel can be reduced to a width of about 100 μm to about 5 μm. In certain embodiments, the channel can be reduced to a width of about 50 μm to about 100 μm, about 55 μm to about 95 μm, about 60 μm to about 90 μm, about 65 μm to about 85 μm, or about 70 μm to about 80 μm. In certain embodiments, the channel can be reduced to a width of about 5 μm to about 80 μm, about 10 μm to about 75 μm, about 15 μm to about 70 μm, about 20 μm to about 65 μm, about 25 μm to about 60 μm, about 30 μm to about 55 μm, about 35 μm to about 50 μm, or about 40 μm to about 45 μm.

In certain embodiments, the cell trapping unit (105) can include a cell inlet (109) in which a fluid (e.g., binding buffer, lysis buffer etc . . . ) or cell suspension can be added to the microfluidic device (100). The fluid or cell suspension can be added to the device by, for example, a syringe pump, a pipette, a tube connected to a cell harvester, or a peristaltic pump. In certain embodiments, the cell trapping unit (105) can be equipped with a cell trapping outlet (110), a cell washing outlet (111), and/or control valves (112). The inlets and outlets of the microfluidic device can be sealed off by plugs. In certain embodiments, the plugs can be made of polycarbonate or any material that is impermeable to water or other liquids.

The cell trapping outlet (110) can allow the fluid or cell suspension to exit the microfluidic device during the cell trapping stage of the process. For example, the buffer carrying the cells enters through the cell inlet (109) and travels through the cell trapping unit (105) and out through the cell trapping outlet (110). Once a cell is trapped, the flow from the cell trap (107) to the cell trapping outlet (110) is stopped. the flow from the cell inlet (109) to the cell washing outlet (111) remains unchanged.

The cell washing outlet (111) can allow excess cells to be washed away once a cell has been trapped. For example, once a cell is trapped, a fluid (e.g., buffer) lacking cells can be added through the cell inlet (109) and pass through the channel and out through the cell washing outlet (111) until no cells remain in the channel except the trapped cell.

The control valves (112) are in the control layer (117) of the microfluidic device. In certain embodiments, the control layer (117) is disposed between the single substrate (115) and the flow layer (118). In certain embodiments, the flow layer (118) is disposed between the single substrate (115) and the control layer (117). In certain embodiments, the control valves (112) can divert and/or direct the flow of the cell suspension for cell trapping and lysis. In certain embodiments, the control valves (112) are pressurized (e.g., hydraulic or pneumatic). In certain embodiments, the control valves utilize pressurized oil to close the valve. The control valve placed before the cell trap (107) is the upstream control valve (112a). The control valve placed after the cell trap (107) is the downstream control valve (112b). The control valves are individually regulated via pressure regulators. In certain embodiments, the control valves interface with the pressure regulators via metal (e.g., stainless steel) and/or plastic (e.g. Tygon) tubing. In certain embodiments, the control valves (112) have the dimensions of about 600 μm to about 1200 μm in length, about 500 μm to about 800 μm in width and about 50 μm to about 150 μm in height. In certain embodiments, the control valves have the dimensions of about 1000 μm in length, about 400 μm in width and about 80 μm in height. In certain embodiments, the control valves (112) have a length of about 600 μm to about 1200 μm, about 700 μm to about 1100 μm, about 800 μm to about 1000 μm, or about 900 μm to about 950 μm. In certain embodiments, the control valves (112) have a width of about 500 μm to about 800 μm, about 550 μm to about 750 μm, about 600 μm to about 700 μm, or about 625 μm to about 650 μm. In certain embodiments, the control valves (112) have a height of about 50 μm to about 150 μm, about 55 μm to about 145 μm, about 60 μm to about 140 μm, about 65 μm to about 135 μm, about 70 μm to about 130 μm, about 75 μm to about 125 μm, about 80 μm to about 120 μm, about 85 μm to about 115 μm, or about 90 μm to about 110 μm.

In certain embodiments, the control layer (117) is below the flow layer (118). In certain embodiments, the control layer (117) is above the flow layer (118).

The microfluidic device can further include microbeads. In certain embodiments, the microbeads are magnetic. The magnetic beads can be moved from one area to the next or held in place via external magnets. The microbeads can be modified to capture genetic material from the lysed cells. The microbeads can be functionalized with, for example but not limited to, primers or small DNA or RNA capture sequences.

An exemplary embodiment of a magnetic microbead (200) for use in the microfluidic device in accordance with the disclosed subject matter is illustrated in FIG. 1B. For example, the magnetic microbead (200) can be functionalized with a primer (201). In certain embodiments, the primer (201) is specifically designed to capture mRNA (202). Once the mRNA (202) is captured, the microbead and attached mRNA can be transported to the reaction chamber to undergo reverse transcription.

In certain embodiments, the bead and reaction reagents can enter through the bead/reagent inlet (114) and travel to the reaction chamber (104), the cell trap (107), or main outlet (113). Any fluids that enter the microfluidic device can exit through the main outlet (113) if they are not precluded as such by the control valves (112) or a plug closing the opening.

### Array

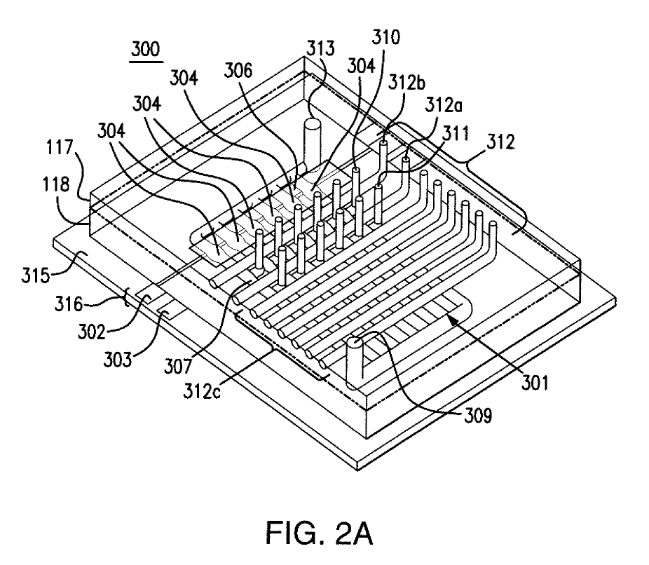

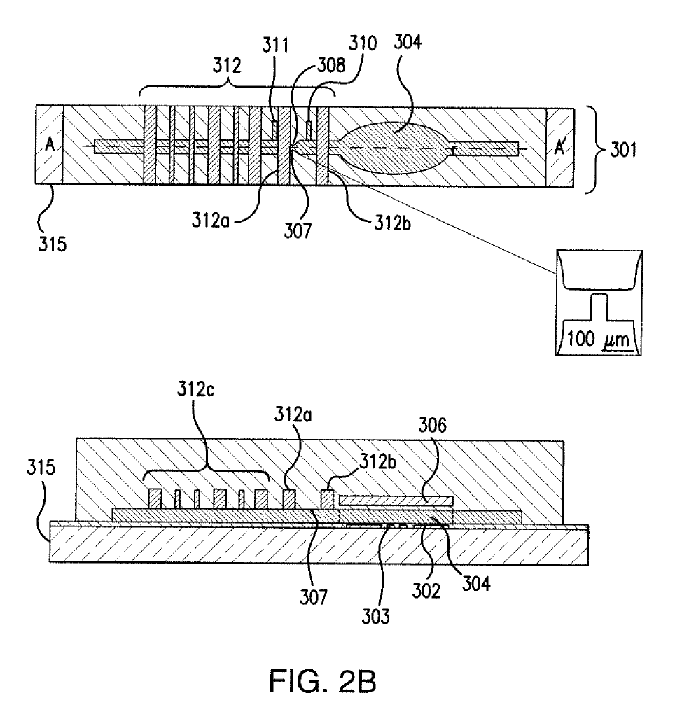

In certain embodiments, the microfluidic device can include more than one analysis unit (301) (i.e., an array). An exemplary embodiment of a microfluidic array (300) in accordance with the disclosed subject matter is illustrated in FIG. 2. As shown in FIG. 2, the microfluidic device (300) can include a temperature control chip with an integrated heater (302) and temperature sensor (303), multiple reaction chambers (304), and multiple cell trapping units (305). The cell trapping unit consists of the valves (312b) and (312a), the cell trap (307), the cell trapping outlet (310), the cell washing outlet (311), and the cell inlet (309). In certain embodiments, the analysis units are identical in design (i.e., repeating identical single analysis units (301)).

The heater (302) and temperature sensor (303) can be integrated beneath the center of the reaction chamber (304). In certain embodiments, the heater (302) and temperature sensor (303) can be integrated between a glass substrate (315) and the flow layer (318). In certain embodiments, the heater (302) and temperature sensor (303) can be integrated between a glass substrate (315) and the control layer (317). Together the temperature sensor (303), heater (302) (i.e., electrodes), and substrate (315) make up the electrode/substrate layer (316).

In certain embodiments, the heater (302) and temperature sensor (303) can be made of chrome (e.g., 10 nm) and gold (e.g., 100 nm) thin films. In certain embodiments, the electrodes can be made of chrome, gold, platinum, aluminum, titanium, or combinations thereof. In certain embodiments, the substrate (315) can be made of a transparent material. The substrate (315) can be made, but not limited to, glass (e.g., a glass slide), clear polymers (e.g., vinyl, acrylic, Plexiglass). In certain embodiments, the substrate (315) is impermeable to water and other liquids.

In certain embodiments, the heater (302) and temperature sensor (303) can be serpentine-shaped. The heater can be, for example, a resistive heater. In certain embodiments, the temperature sensor 3) has a linewidth of about 20 μm to about 100 μm. In certain embodiments, the temperature sensor (303) has a linewidth of about 50 μm. In certain embodiments, the temperature sensor (303) has a linewidth of about 25 μm to about 95 μm, about 30 μm to about 90 μm, about 35 μm to about 85 μm, about 40 μm to about 80 μm, about 45 μm to about 75 μm, about 50 μm to about 70 μm, or about 55 μm to about 65 μm. In certain embodiments, the heater (302) has a linewidth of about 200 μm to about 1000 μm. In certain embodiments, the heater (302) has a linewidth of about 400 μm. In certain embodiments, the heater (302) has a linewidth of about 250 μm to about 950 μm, about 300 μm to about 900 μm, about 350 μm to about 850 μm, about 400 μm to about 800 μm, about 450 μm to about 750 μm, about 500 μm to about 700 μm, or about 550 μm to about 650 μm.

The reaction chamber (304) is in the flow layer (318) of the microfluidic device. In certain embodiments, the reaction chamber (304) is designed for a two-step or three-step RT-qPCR process. In certain embodiments, the reaction chamber (304) can be made of polydimethylsiloxane (PDMS). The reaction chamber (304) can be elliptical, cylindrical, or rectangular with a trapezoid on the top and bottom. In certain embodiments, the reaction chamber (304) can be any shape with a flat bottom. In certain embodiments, the reaction chamber (304) can be elliptical with the dimensions of about 5 mm to about 10 mm in length, about 2 mm to about 6 mm in width, and about 10 μm to about 25 μm in height. In certain embodiments, the reaction chamber (304) is elliptical with the dimensions of about 7.7 mm in length, about 5.7 mm in width, and about 15 μm in height. In certain embodiments, the reaction chamber (304) can have the dimensions of about 5 mm to about 10 mm in length, about 6 mm to about 9 mm in length, or about 7 mm to about 8 mm in length. In certain embodiments, the reaction chamber (304) can have the dimensions of about 2 mm to about 6 mm in width, about 2.5 mm to about 5.5 mm in width, about 3 mm to about 5 mm in width, or about 3.5 mm to about 4.5 mm in width. In certain embodiments, the reaction chamber (304) can have the dimensions of about 10 μm to about 25 μm in height, about 12.5 μm to about 22.5 μm in height, or about 15 μm to about 20 μm in height. In certain embodiments, the reaction chamber (304) has a volume of about 100 nL to about 1500 nL. In certain embodiments, the reaction chamber (304) has a volume of about 655 nL. In certain embodiments, the reaction chamber (304) has a volume of about 100 nL to about 1500 nL, about 200 nL to about 1400 nL, about 300 nL to about 1300 nL, about 400 nL to about 1200 nL, about 500 nL to about 1100 nL, about 600 nL to about 1000 nL, or about 700 nL to about 900 nL.

In certain embodiments, the reaction chamber (304) is covered with an evaporation barrier (306). The evaporation barrier (306) is made of the same materials and in the same fashion as described above with the single analysis unit. In certain embodiments, the evaporation barrier (306) can be configured to cover all of the reaction chambers (304) of the array's analysis units. In certain embodiments, the evaporation barrier (306) can cover each reaction chamber (304) separately.

The microfluidic array can include multiple cell trapping units (305) in the flow layer (318). In certain embodiments, the microfluidic array can include a single cell trapping unit (305) that leads into different reaction chambers (304). In certain embodiments, the cell trapping unit (305) can have the dimensions of about 500 μm to about 2000 μm in length, about 80 μm to about 120 μm in width, and about 10 μm to about 25 μm in height. In certain embodiments, the cell trapping unit (305) can have the dimensions of about 800 μm in length, about 100 μm in width and about 15 μm in height. In certain embodiments, the cell trapping unit (305) can have the length of about 500 μm to about 2000 μm in length, about 600 μm to about 1900 μm, about 700 μm to about 1800 μm, about 800 μm to about 1700 μm, about 900 μm to about 1600 μm, about 800 μm to about 1500 μm, about 900 μm to about 1400 μm, about 1000 μm to about 1300 μm, or about 1100 μm to about 1200 μm. In certain embodiments, the cell trapping unit (305) can have the width of about 80 μm to about 120 μm, about 85 μm to about 115 μm, about 90 μm to about 110 μm, or about 95 μm to about 105 μm. In certain embodiments, the cell trapping unit (305) can have the height of about 10 μm to about 25 μm, about 11 μm to about 24 μm, about 12 μm to about 23 μm, about 13 μm to about 22 μm, about 14 μm to about 22 μm, about 15 μm to about 21 μm, about 16 μm to about 20 μm, or about 17 μm to about 19 μm.

Each cell trapping unit (305) can include a cell trap (307). In certain embodiments, the cell trap (307) can be a neck-shaped channel. The cell trap (307) can be created by a narrowing of the channel (308). The narrowing in the channel can reduce the channel width to smaller than the average diameter of the cell type to be trapped. In certain embodiments, the channel can be reduced from a width from about 80 μm to about 120 μm down to about 4 μm to about 5 μm. In certain embodiments, the channel can be reduced to a width of about 100 μm to about 5 μm. In certain embodiments, the channel can be reduced to a width of about 50 μm to about 100 μm, about 55 μm to about 95 μm, about 60 μm to about 90 μm, about 65 μm to about 85 μm, or about 70 μm to about 80 μm. In certain embodiments, the channel can be reduced to a width of about 5 μm to about 80 μm, about 10 μm to about 75 μm, about 15 μm to about 70 μm, about 20 μm to about 65 μm, about 25 μm to about 60 μm, about 30 μm to about 55 μm, about 35 μm to about 50 μm, or about 40 μm to about 45 μm. In certain embodiments, the protruding structure (i.e., the portion of the mold that narrows the channel) can reduce the channel width from about 100 μm to about 5 μm (i.e., a narrowing of the channel).

The cell trapping unit (305) can include a main inlet (309) in which a fluid (e.g., binding buffer, lysis buffer etc . . . ) or cell suspension can be added to the device (300). The fluid or cell suspension can be added to the device by, for example, a syringe pump, a pipette, or a tube connected to a cell harvester. In certain embodiments, the cell trapping unit (305) can be equipped with a cell trapping outlet (310), a cell washing outlet (311), and/or control channels or valves (312). The inlets and outlets of the microfluidic device can be sealed off by plugs. In certain embodiments, the control valves (312) are pressurized (e.g., hydraulic or pneumatic). In certain embodiments, the control valves utilize pressurized oil to close the valve.

The cell trapping outlet (310) can allow a fluid or cell suspension to exit the microfluidic device during the cell trapping stage of the process. For example, the buffer carrying the cells enters through the main inlet (309) and travels through the cell trapping unit (305) and out through the cell trapping outlet (310).

The cell washing outlet (311) can allow excess cells to be washed away once a cell has been trapped. For example, once a cell is trapped, a fluid (e.g., buffer) lacking cells can be added through the main inlet (309) and pass through the channel and out through the cell washing outlet (311) until no cells remain in the channel except the trapped cell.

The control valves are in the control layer (317) of the microfluidic device. In certain embodiment, the control layer (317) is disposed on top of the flow layer (318). In certain embodiment, the flow layer (318) is disposed on top of the control layer (317). In certain embodiments, the control channels or valves (312) can divert the flow of the cell suspension for cell trapping and lysis. The control valve placed before the cell trap (307) is the upstream control valve (312a). The control valve placed after the cell trap (307) is the downstream control valve (312b). In certain embodiments, the control valves (312) have the dimensions of about 300 μm to about 600 μm in length, about 200 μm to about 500 μm in width and about 10 μm to about 25 μm in height. In certain embodiments, the control valves (312) have a length of about 300 μm to about 600 μm, about 350 μm to about 550 μm, or about 400 μm to about 450 μm. In certain embodiments, the control valves (312) have a width of about 200 μm to about 500 μm, about 250 μm to about 450 μm, or about 300 μm to about 400 μm. In certain embodiments, the control valves (312) have a height of about 10 μm to about 25 μm, about 12 μm to about 24 μm, about 14 μm to about 22 μm, about 16 μm to about 20 μm, or about 17 μm to about 18 μm.

The microfluidic array can have multiple control valves (312). In addition to the two control valves on either side of the cell traps (307) (i.e., cell trapping valves (312a, 312b)), the device can contain an additional control valve for every analysis unit of the array (i.e., multiplexing valves (312c)). For example, the microfluidic array of FIG. 2 contains six analysis units; therefore, in addition to the two cell trapping valves (312a, 312b), the microdevice contains six multiplexing valves (312c). The multiplexing valves can allow for precise control of flow within a single analysis unit while mutually preventing flow in the other five analysis units. The control valves are regulated in the same manner as discussed above with the single analysis unit.

The microbeads have the same specifications as discussed above with the single analysis unit. In certain embodiments, the bead/and reaction reagents can enter through the main inlet (309). In certain embodiments, the array has an independent inlet for each array unit. and travel to the reaction chamber (304) or main outlet (313). Any fluids that enter the microfluidic device can exit through the main outlet (313) if they are not precluded as such by the control valves (312).

### Fabrication of the Microfluidic Devices

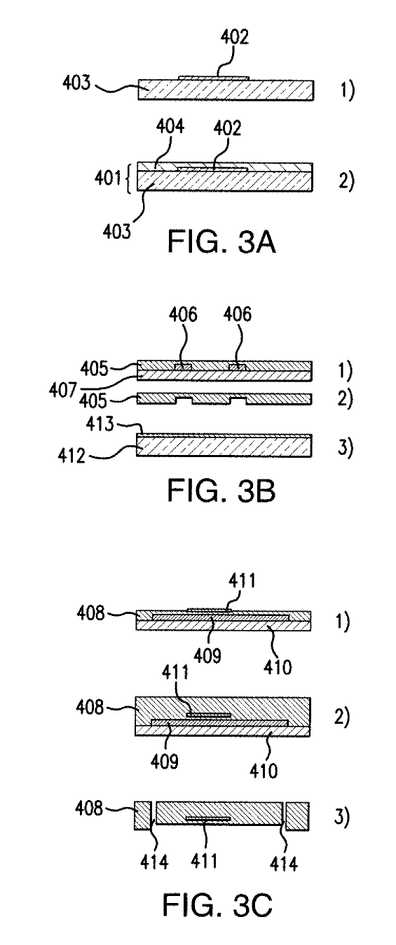

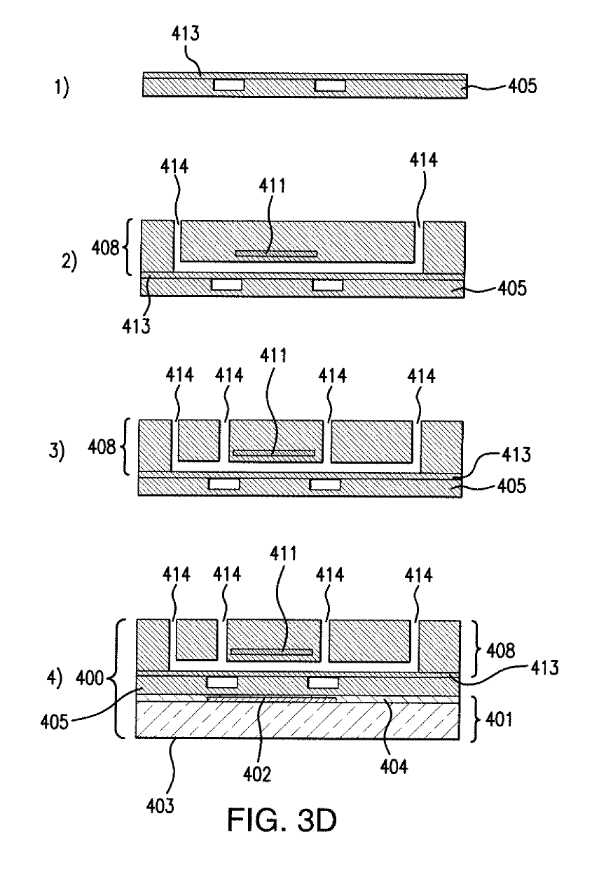

The microfluidic device can be fabricated using multi-layer soft lithography microfabrication techniques. An exemplary embodiment of the fabrication of a microfluidic device (400) in accordance with the disclosed subject matter is illustrated in FIG. 3. As discussed in greater detail below, the process outlined in FIG. 3 demonstrates: (3A) heater and sensor formation steps of 1) electrode (e.g., Au/Cr) deposition and 2) passivation; (3B) control layer and membrane formation steps of 1) control layer mold fabrication using standard soft lithography and PDMS pouring; 2) PDMS baking and peeling off; and 3) featureless PDMS membrane spin coating; (3C) flow layer formation steps of 1) mold fabrication, PDMS baking and evaporation barrier aligning; 2) evaporation barrier implantation; and 3) flow layer release and holes punching; and (3D) device package using RIE method.

The microfluidic device (400) contains an electrode/substrate layer (401), a control layer (405), and a flow layer (408). In certain embodiments, the control layer (405) is disposed between the electrode/substrate layer (401) and the flow layer (408). In certain embodiments, the flow layer (408) is disposed between the electrode/substrate layer (401) and the control layer (405).

The electrode/substrate layer (401) contains the temperature chip. In certain embodiments, electrodes (402) can be deposited and patterned onto a substrate (403) (e.g., glass slide). In certain embodiments, the electrode/substrate layer (401) can be reusable. The electrodes (402) form the heater (e.g., 102, 302) and temperature sensor (e.g., 103, 303) of the microfluidic device (400). In certain embodiments, the electrode can be made of chrome (e.g., 10 nm) and gold (e.g., 100 nm) thin films. In certain embodiments, the thickness of the film, can vary. For example, the film can be from about 10 nM to about 500 nM. In certain embodiments, the thickness of the film can be about 50 nM to about 450 nM, about 100 nM to about 400 nM, about 150 nM to about 350 nM, or about 200 nM to about 300 nM. In certain embodiments, the film can be about 10 nM to about 100 nM, about 20 nM to about 90 nM, about 30 nM to about 80 nM, about 40 nM to about 70 nM, or about 50 nM to about 60 nM. In certain embodiments, the film can be about 100 nM to about 500 nM, about 125 nM to about 475 nM, about 150 nM to about 450 nM, about 175 nM to about 425 nM, about 200 nM to about 400 nM, about 225 nM to about 375 nM, about 250 nM to about 350 nM, or about 275 nM to about 325 nM. In certain embodiments, the film is about 10 nM, about 80 nM, or about 100 nM. In certain embodiments, the film thickness is at least about 10 nM, at least about 50 nM, at least about 80 nM, or at least about 100 nM. In certain embodiments, the electrodes can be made of chrome, gold, platinum, aluminum, titanium, or combinations thereof. Next, the electrode layer is passivated (404). The method of passivation can include, but is not limited to, sequential spin coating and curing of a layer of a PDMS, or chemical vapor deposition of silicon dioxide, or parylene deposition. In certain embodiments, the parylene deposition is followed by spin coating PDMS and baking the electrode layer.

The control layer (405) contains the control valves (e.g., 112, 312). Molds (406) for the control valves can be fabricated by spin coating a photoresist in a pattern onto a substrate (407) (e.g., piranha cleaned silicon substrate) and developed. In certain embodiments, the photoresist for the molds (406) of the control layer (405) can be SU-8, AZ photoresists, or like material. The size of the molds (406) can be measured using a profilometer (e.g. Dektak 3). The material of the control layer (e.g., PDMS) can be poured over the mold and developed.

The flow layer (408) contains the reaction chamber(s) (e.g., 104, 304), the evaporation barrier (e.g., 106, 306) cell trapping unit (e.g., 105, 305), the inlets (e.g., 109, 112, 309), and the outlets (e.g., 110, 111, 113, 313). Molds (409) for the reaction chamber(s), cell trapping unit(s), and channels connecting each to each other and the inlets and outlets can be fabricated by spin coating a photoresist in the appropriate pattern on a substrate (410). In certain embodiments, the substrate (410) can be silicon. In certain embodiments, the photoresist for the molds (409) of the flow layer (408) can be AZ photoresist (e.g., AZ 4620) and/or SU-8. The size of the molds (406) can be measured using a profilometer (e.g. Dektak 3). For example, AZ 4620 photoresist can be spun coated and patterned. Once developed, the photoresist can be heated above the glass transition temperature of the photoresist, which results in the reflowing of the photoresist formed channels with a rounded cross section. This is done prior to the PDMS being poured. In certain embodiments, PDMS can be poured over the flow layer molds (409). In certain embodiments, an evaporation barrier (411) can be embedded in the flow layer (408). In certain embodiments, the evaporation barrier (411) is bonded to the top of the reaction chamber(s) (e.g., 104, 304). For example, but not by way of limitation, a two-step PDMS casting process can be used to embed the evaporation barrier (411) above the reaction chamber(s) (e.g., 104, 304). For example, the base and agent of PDMS can be mixed (e.g. at a ratio of 11:1, 10:1 or 9:1) and spun coated on the mold (409) followed by the stamping of an adhesive film on the PDMS at the region of the reaction chamber (e.g., FIG. 3 C, step 1) followed by an additional layer of PDMS backed on top (e.g., FIG. 3 C, step 2).

In certain embodiments, uncured PDMS was spun on a wafer (412) to form a featureless membrane (413).

Sheets bearing the microfluidic features (e.g., 405, 408, and 413) are peeled off the molds (e.g., 406, 409) followed by inlet and outlet hole (414) punching. In certain embodiments, the holes in the flow layer (408) can be punched with a mechanical punch. The membrane (413) can be sandwiched between the flow (408) and control (405) layers by oxygen plasma. Finally, the control layer (405), membrane (413) and flow layer (408) can be bonded to the electrode/substrate layer (401) by oxygen plasma resulting in a packaged microfluidic device (400).

### Transcriptional Profiling Method

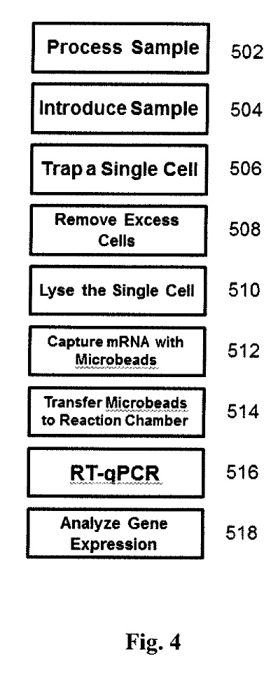

In one aspect, the presently disclosed subject matter provides a method for immobilizing, lysing, and performing transcriptional profiling analysis of a single cell. Referring to FIG. 4, the method can include processing a sample (502). The sample is processed to break up cell clusters until a cell suspension is formed. Once processed, the cells can, for example, be diluted to about 10⁴ cells/ml, about 10⁵ cells/ml, about 10⁶ cells/ml, about 10⁷ cells/ml, about 10⁸ cells/ml, about 10⁹ cells/ml, or about 10¹⁰ cells/ml in an appropriate buffer system. Dilution of the cells can allow for efficient trapping of a single cell rather than blocking the cell trap with a high density of cells.

In certain embodiments, the methods can be applied to gene regulation studies by detecting drug induced and/or diseased induced changes in gene expression in a single cell. In certain embodiments, the methods can be applied to studies on the environmental effects on cells. In certain embodiments, the methods can be applied to studies on radio induced changes on cells.

The sample can be derived from a bodily fluid, a tissue sample, or cell culture. In certain embodiments, the bodily fluid, a tissue sample, or cells can be obtained from a human or animal. In certain embodiments, the cells can be healthy cells. In certain embodiments, the cells can be diseased cells. In certain embodiments, it is unknown whether the cells are healthy or diseased. In certain embodiments, the cells can be cancer cells.

In certain embodiments, this method can be applied to various bodily fluids, including but not limited to, tears, blood, saliva, mucus, interstitial fluid, spinal fluid, intestinal fluid, amniotic fluid, lymphatic fluid, pericardial fluid, peritoneal fluid, pleural fluid, semen, vaginal secretions, sweat, and synovial fluid of the subject.

In certain embodiments, this method can be applied to various tissues, including tissues from any part of the body, not limited to, arteries, bladder, blood, brain, breast, capillary beds, cervix, colon, cornea, eye retina, gastrointestinal tract, gynecological tract, hair, heart, intestines, kidney, liver, lung, muscle, ovary, prostate, retinal blood vessel, skin, stomach, tumor, veins, and combinations thereof.

In certain embodiments, this method can be applied to various cell cultures, including but not limited to, Primary-hBM SC; Primary-hSkin FB; Primary-cow CC; Primary-rat BMSC; Primary-h CC; MC3T3-E1; Primary-hUVEC; Primary-rabbit CC; NIH 3T3; Primary-CC; Primary-rat Liver Hep; Primary-hSkin Keratinocyte; MG63; HEP-G2; L929; Primary-BM SC; Primary-rabbit BM SC; Primary-pig CC; Primary-hBone OB; MCF-7; Primary-rat Heart CM; Primary-h Foreskin FB; Primary-hAdipose SC; Primary-hFB; #N/A; Primary-hAdipose SC; Primary-FB; Primary-ratAortaSMC; Primary-Bone; Primary-dog CC; 3T3 (nonspecific); C2C12; MDA-MB-231; SaOS-2; Primary-mouse BM SC; Primary-rat CC; Primary-h Mesoderm Mes Pre C; Primary-rat Brain Neuronal; PC12; Primary-Cancerous; Primary-h Skin EC; Primary-rat BM OB; Primary-mouse Embryo SC; MCF-10A; Primary-h Bone OB-like; Primary-goat BMSC; Primary-h Aorta SMC; MDCK (Madin-Darby Canine Kidney); Primary-hi DAnnulus C; Primary-ratBone OB; Primary-h Adipose Preadipocyte; Primary-SC; Primary-rat Skeletal Muscle Myoblast; Primary-Heart CM; Primary-cow AortaEC; Primary-dog BM SC; Primary-sheep BM SC; Primary-sheep CC; Primary-pig BMSC; Primary-cow BMSC; Primary-h BladderSMC; Primary-pig Aorta EC; Primary-h Cornea Epi C; Primary-h Aorta EC; Primary-h Cornea FB; Primary-pig Aorta SMC; Primary-mouse Liver Hep; A549; Primary-Bone OB; Primary-h Bladder Uro; Primary-h UV SMC; Swiss 3T3; Primary-Liver Hep; Primary-h Lig FB; Primary-h Coronary Artery SMC; Primary-OB-like; Primary-h Teeth Mes Pre C; HT1080; Primary-rat Heart FB; Primary-pig HV Intersticial C; C3A; Primary-h Breast Cancerous; Primary-h Foreskin Keratinocyte; Primary-h Oral Mucosa Keratinocyte; Primary-mouse Ovary Oocytes; Primary-h Vase SMC; 3T3-L1; Primary-h Lung FB; Primary-chicken Ganglia Neuronal; Primary-h U CStC; Primary-cow Aorta SMC; Primary-mouse Embryo FB; Primary-h Bronchi EpiC; CHO-K1; Primary-h Liver Hep; Primary-hSaphVEC; Primary-hTeethPDL; Primary-rat Skin FB; Primary-pig Liver Hep; PC-3; Primary-SMC; Primary-hMVEC; Primary-mouseFB; Primary-h Nasal Chondrocyte; Primary-hCorneaKeratinocyte; Primary-hOvaryCancerous; Primary-h U CBSC; Primary-rat Heart EC; Primary-Vasc; Primary-mouse Skin FB; Primary-h Tendon TC; Primary-rat Brain Astrocyte; Primary-rat Nerve SC; Ha CaT; Primary-h Gingiva FB; Primary-Neural; Primary-cow Bone OB; Primary-rat Adipose SC; Primary-mouse Bone OB; Primary-h Teeth PC; Primary-h Blood Mononuclear; Primary-rat Hippocampus Neuronal; D3; HeLa; HEK293; C17.2; Primary-h Skin Melanocyte; Primary-h Blood EC-like; HOSTE85; Primary-h UC SC-like; Primary-h Cornea SC; Primary-rat Aorta EC; Primary-h Saph VSMC; Primary-h UCBEC; Primary-mouse Heart CM; D10RL UVA; Primary-h Coronary Artery EC; Primary-h Aorta Myo FB; HT-29; Primary-h Tendon FB; RAW 264; Primary-rat Dental Pulp SC; 3T3-J2; H1; Primary-pig Teeth; Primary-rat Sciatic Schwann; Primary-rabbit Bone OB-like; Primary-sheep Aorta EC; Primary-rabbit Cornea Epi C; Primary-h Ovary Epi C; Primary-rabbit Ear Chondrocyte; SH-SYSY; Primary-h Teeth FB; Primary-h Oral Mucosa FB; Primary-rabbit FB; C6; Primary-rat Testes Stertoli; Primary-cow Arterial EC; Primary-pigHVEC; Primary-cow Nucleus Pulposus Cells; Primary-rat Ganglia Neuronal; Primary-dog Bladder SMC; Primary-Vasc SMC; 129/SV; Primary-pig Ear Chondrocyte; ED27; Primary-rabbit Bone B; Primary-h Brain Glioblast; Primary-rat Adipose Preadipocyte; Primary-h Cartilage Synov; Primary-rat Pancreas Insulin; Primary-hEC; Primary-sheep Aorta SMC; Primary-h Endometrium EpiC; U251; Primary-h Endometrium StC; Primary-pig Bladder SMC; Primary-h HVIintersticial C; Primary-pig Esoph SMC; Primary-h NP Neuronal; Primary-rabbit Aorta SMC; Primary-h NSC; Primary-rabbit CorneaFB; Primary-h ral Cancerous; Primary-rabbit Lig FB; Primary-h SC; Primary-rat BMOB-like; Primary-h Skeletal Muscle Myoblast; COS-7; C-28/12; HK-2; Primary-h Uterus Cancerous; Primary-rat Ventricle CM; Primary-h Vase EC; Primary-sheep Carotid Artery SMC; HCT-116; ROS 17/2.8; Primary-h Vocal FB; UMR-106; Primary-mouse Aorta SMC; H9; R1; Primary-rat Fetal Neuronal; Primary-chicken Ear EpiC; Huh7; Primary-rat Vasc SMC; Primary-h NP SC; ES-D3; IMR-90; Primary-rat Bladder SMC; 293T; Primary-h Foreskin VascularEC; Primary-h Placenta EC; Primary-h Lung EpiC; Primary-h Prostate EpiC; U-87 MG; Primary-dog Carotid Artery SMC; Primary-rabbit Cornea StC; Primary-dog ID Annulus Fibrosus; Primary-chicken Embryo Chondrocyte; Primary-EC; HFF; Vero; HFL-1; Primary-h Adipose FB; Primary-cow FB; Primary-h UTSMC; Primary-rat Ventricle FB; AH 927; Primary-sheep Vasc FB; DU-145; ST2; B16.F10; Primary-h Nasal EpiC; Primary-ID Annulus C; Primary-h Dental Pulp SC; 3H10T1/2; Primary-Heart Valve; Primary-h Bone Alveolar; Primary-rabbit Tendon FB; Primary-mouse Kidney Insulin; HEPM; Primary-baboon Aorta SMC; HTK; Primary-mouse MDSC; Primary-rat Esoph EpiC; Primary-mouse Nerve SC; Primary-h Fetus OB-like; Primary-mouse Skeletal Muscle SC; hFOB 1.19; Primary-Nerve Schwann; Primary-h Ganglia Neuronal; Caco-2; Primary-h Kidney Renal; Primary-h Breast EpiC; Primary-h Liver SC; Primary-pig Bladder Uro; Primary-h Lung EC; Primary-h Breast FB; Primary-sheep Jugular Vein EC; Primary-pig Esoph EpiC; Primary-h Lymph EC; Primary-chicken CC; Primary-h Lymph TCell; Primary-h Colon Adenocarcinoma; Primary-h Mammary EC; Primary-pig Vocal FB; Primary-h Mammary EpiC; Primary-rabbit Adipose SC; Primary-h Cornea EC; H9c2; Primary-h UT StC; Primary-cat Heart CM; Primary-mouse Pancreas EpiC; HS-5; Primary-sheep Skeletal Muscle Fetus Myoblast; Primary-cow ID; Primary-mouse BM OCpre; Primary-cow Knee Meniscus C; Hep-3B; Primary-cow Lig FB; HL-1; HuS-E/2; RWPE1; Primary-cow Retina EpiC; Primary-hVascMyoFB; IEC-6; Primary-mouse Fetal Hep; HS68; OVCAR-3; Primary-dog Knee MeniscusC; Primary-rabbit Mesoderm Mes PreC; Primary-dog Lig FB; Primary-rat Lung Alveolar; Primary-dog Skin Keratinocyte; CRL-11372; Primary-dog Vase SMC; HMEC-1; Primary-Embryo SC; T-47D1; Pimary-goatCC; Primary-h UVSC-like; Primary-guineapig Ear EpiC; Primary-Ligament; Primary-guineapig Skin FB; Primary-mouse Cortical Neuronal; Primary-hAdipose Adipocyte; Primary-mouse Liver SC; Primary-h Adipose FB-like; CAL72; J774; P19; Primary-h Amniotic fluid; Primary-rabbit Cornea EC; Primary-h Amniotic FSC; Primary-rat BMFB-like; ARPE-19; Primary-rat Kidney Mesangial; K-562; Primary-rat Nasal Ensheathing; Primary-h Bladder StC; Primary-chicken Embryo Proepicardium; ATDC5; Primary-sheep FB; Kasumi-1; Primary-Skeletal Muscle; Primary-h Bone Mes PreC; HMT-3522; Primary-h Bone Periosteal; A431; Primary-h Brain EC; Primary-h UTFB; KLE; 143b OST; BALB/3T3; Primary-h Vasc FB; LLC-PKI; Primary-h Vasc Pericyte; BHK21-C13; Primary-Mammary EpiC; M.DUNNI; C4-2B; ZR-75; HEC-1B; Primary-h Gingiva Keratinocyte; U178; Primary-h HN Cancerous; Primary-mouse Mammary EpiC; Primary-h Keratinocyte; Primary-mouse Sciatic N Schwann; OVCA429; Primary-h Kidney EpiC; Primary-pig Esoph FB; MBA-15; Primary-pig Mandible FB-like; Primary-h Liver Cancerous; Primary-rabbit Bladder Uro; GD25betalA; Primary-rabbit ID AnnulusC; HSC-T6; Primary-rabbit NP Neuronal; DOV13; HEY; Primary-h Mammary FB; HTB-94; BZR-T33; Primary-chicken CorneaFB; MiaPaCa2; Primary-rat Mucosa Ensheathing; Primary-hOvaryFB; Primary-rat Salivary Acinar; Primary-h Ovary Oocyte; Primary-rat Testes Germ; Primary-h Pancreas Cancerous; Primary-chicken Embryo StC; Primary-h Pancreas Stellate Cells; Primary-sheep Carotid Artery FB; ML0-Y4; Primary-chicken Retina SC-like; Primary-h Prostate Cancerous; Primary-chicken Ten TC; Primary-h Saph V Myo FB; Primary-Synoviocyte; MTLn3; Primary-Vasc EC; Primary-h Skeletal Muscle Pre; RT4-D6P2T; C2; SCA-9; HOC-7; T31; Primary-h UC EpiC; TR146; HCS-2/8; EA.hy926; Primary-rat Ebryo; SW480; Primary-sheep Fetus CC; Primary-dog Pancreas Insulin; KS-IMM; BPH-1; Primary-rat Pancreas SC; M2139; RIN-5F; Primary-hGallbladderCancerous; E14/TG2a; M4E; HES3; G8; Primary-hConjunctivaFB; Primary-dogSaphVEC; LN CaP; Primary-dog Saph V SMC; M4T; Primary-h Fetus CC; BR-5; Primary-pig UT Uro; Primary-Hippocampus Neuronal; PE-0041; Primary-dog Skin FB; Primary-rabbit Skeletal Muscle MyoBlast; Primary-cow Denta ipulp; CGR8; Primary-dog Teeth PDL; Primary-rat Fetus Hep; Primary-dog Tendon FB; Primary-rat Mammary; Primary-h Knee C; Primary-rat SMC; BRC6; Primary-sheep Artery FB; Primary-dog Vasc EC; Primary-cow Mammary Alveolar; pZIP; 293 cell line; BMC9; Primary-h Lung Cancerous; SKOV-3; IOSE; TEC3; MCF-12A; Primary-rabbitBladderEpiC; Gli36DeltaEGFR; Primary-rabbit Conjunctiva EpiC; Primary-h Lung Neuronal; Primary-rabbit Endometrium EpiC; 1205Lu; Primary-rabbit MDSC; 3T3-A31; Primary-rabbit Tendon Tenocyte; MDA-MB-435; Primary-h Cancerous; Primary-cow EC; Primary-rat Cornea FB; Primary-EpiC; Primary-rat Fetal Cardiac; Primary-h Meninges Arachnoidal; COS-1; Primary-Eye; Primary-rat Liver Oval C; GLUTag-INS; Primary-rat Oral Mucosa Keratinocyte; GM3348; CRFK; 21NT; Primary-rat Testes EC; Primary-h Nasal FB; Primary-h Dura MaterSC; Primary-h Nasal OB; Primary-dog NP Neuronal; Primary-h Nasal Secretory; Primary-sheep Lung FB; AC-1M59; BHPrE1; MING; Primary-UT; MKN28; RAT-2; MLO-A5; RT112; CRL-2266; S91; GM5387; SK-ChA-1; Primary-horse CC; SPL201; Primary-horse Tendon FB; Primary-h Fetus Mes PreC; D283; Primary-pig Thyroid EpiC; H1299; Par-C10; AE-6; Primary-rabbit Blood Platelet; Primary-goat Carotid EC; Primary-rabbit Bone OC; Primary-goat Carotid FB; Primary-cow Cornea FB-like; Primary-h Pancreas SC; Primary-rabbit CT Pericyte; Primary-goat Carotid SMC; Primary-rabbit Esophagus SMC; Primary-h Parotid Acinar; Primary-baboon Blood EC; A498; Primary-h Bronchi SMC; Primary-h Placenta SC; Primary-rabbit Sphincter SMC; Primary-cow Retina SC; 7F2; MM-Sv/HP; A10; Primary-h Prostate StC; Primary-buffalo Embryo SC-like; Primary-h Salivary Cancerous; CHO-4; Primary-h Salivary Salisphere; Primary-rat Cortical Neuronal; H13; Primary-rat Embryo Neuronal; Primary-guineapig Pancreas EpiC; Primary-rat Fetal OB; H144; CNE-2; MPC-11; 21PT; Primary-cow Synovium; Primary-rat Liver EC; Primary-cow Fetus CC; BEAS-2B; H2122; LM2-4; Detroit 551; C18-4; FLC4; Ishikawa; Primary-rat Skin Keratinocyte; H35; Primary-rat Tendon; Primary-h SMC; HTR8; Primary-h Synovial CC; E8.5; H460M; HL-60; MUM-2B; CRL-1213; MUM-2C; CRL-12424; W20-17; Lovo; Primary-dog Blood EC; Primary-sheep Nasal CC; HAK-2; Primary-sheep Skin FB; Primary-h Testes Sertoli; Primary-h Thyroid Cancerous; Primary-Trachea; Primary-h Trachea; LRM55; Primary-h UASC-like; Primary-Colon FB; Primary-hUASMC; r-CHO; HAT-7; RN22; HC-11; Primary-h Eye Vitreous; AEC2; S2-020; HCC1937; CRL-2020; AG1522; SCC-71; N18-RE-105; SK-N-AS; Primary-h Uterus SMC; SLMT-1; IMR-32; STO; NB4; Swan 71; Primary-h Alveolar Perosteum; Primary-dog Oral Mucosa EpiC; Primary-h Amnion EP; Primary-h Fetus Schwann; Primary-dog Bone OB; Primary-pig UTSMC; 184A1; Panc 1; NCTC 2544; 46C; Primary-cow Cornea EC; B6-RPE07; Primary-hamster EC; cBAL111; Primary-hamster Retina Neuronal; HEPA-1C1c7; NEB1; CCE; NHPrE1; Primary-rabbit Conjunctiva FB; 410; Hepa RG; Primary-Keratinocyte; PMC42-LA; Primary-dog Cartilage Synov; 21MT; NOR-P1; Primary-rabbit Endometrium StC; Primary-Lymphnode Lymphocyte; DLD-1; Primary-Lymphnode TCell; Primary-rabbit Lacrimal Gland Acinar; AB2.1; primary-rabbit Lung Pneumocyte; Primary-monkey Embryo; ES-2; Primary-monkey Kidney FB-like; Primary-rabbit Penis SMC; Primary-mouse Adipose StC; Primary-rabbit Skin FB; NR6; Primary-Blood SC; Primary-mouse BM Macrophage; 786-0; AT2; Primary-rat Adrenal Chromaffin; AT3; CCF-STTGI; Primary-mouse Bone Calvarial; Primary-rat Bladder Uro; HCT-8/E11; CE3; Primary-mouse Brain Neuronal; CFK2; Primary-mouse Breast Cancerous; L6; Primary-mouse Chondrocytes; HeyA8; Primary-mouse Colon EpiC; Primary-rat Cortical Astrocyte; Primary-dog CFB; Primary-buffalo Ovary EpiC; Primary-dog Cornea Chondrocyte; Primary-rat Embryo CM; Primary-mouse Embryo Neuronal; A2780; C5.18; Primary-dog MV EpiC; Primary-mouse Esophagus SC; Primary-rat Fetal Renal; HEK001; A357; EFO-27; Primary-chicken Bone OB; Primary-mouse Fetal Lung; Primary-rat Heart SC-like; Primary-mouse Germ; Primary-rat Kidney; EN Stem-A™; Primary-rat Lacrimal Acinar; U-251 MG; Primary-dog Myofibroblasts; A4-4; Primary-rat Liver SC-like; Primary-cow Brain EC; Primary-rat Lung FB; Primary-mouse Kidney Renal; BEL-7402; NT2; HIAE-101; Primary-h BM Mononuclear; Primary-rat Ovary; Primary-mouse Lymph FB-like; Primary-rat Pancreas Islets; Primary-dog Esophageal EpiC; Primary-rat Renal EpiC; Primary-mouse Mast; Primary-chicken Embryo Blastoderm; NTera2/cl.D1; G-415; Null; Primary-rat Small Intestine; Primary-mouse Ovary Cumulus C; Primary-rat Teeth SC-like; HEL-299; Primary-rat Tendon Tenocyte; KB; b-End-2; Primary-mouse Pancreas Insulin; Primary-rat Vase EC; Primary-mouse Salivary Salisphere; Primary-h Duodenum EpiC; Primary-h Bone Fetus OB; Primary-Respiratory EpiC; Primary-mouse Skeletal Muscle Myoblast; Primary-sheep Amniotic fluid; OC2; Primary-chicken Heart CM; Daudi; Primary-shee pArtery MyoFB; Primary-mouse SkinKeratinocyte; Primary-sheep Bone OB-like; Primary-mouse Small Intestine; Primary-chicken Heart ECM; Primary-mouse Spleen Tcell; LNZ308; Primary-mouse Teeth Odontoblast; Primary-sheep ID Annulus Fibrosus; Primary-mouse Testes SC; Primary-sheep Jugular Vein SMC; Primary-mouse Testes Sperm; Primary-sheep Lung SC; Primary-mouse UT Uro; Primary-sheep Saph VEC; Primary-mouse Uterus EpiC; Primary-sheep Skin EC; OCT-1; Primary-sheep Vasc EC; HELF; Primary-sheep Vasc SMC; CAC2; HL-7720; OPC1; Primary-Teeth PDL; Primary-dog Heart SC; Primary-UCB Mononuclear; Primary-pig Artery Carotid EC; Primary-h Endometriotic CystStC; Primary-pig Artery Carotid SMC; Primary-Colon Cancerous; Primary-pig Artery Coronary SMC; QCE-6; Primary-pig Bladder FB; R221A; OSCORT; LS180; B35; RIF-1; Calu-1; RL-65; Calu-3; Primary-cow Adrenal ChrC; B5/EGFP; RT-112; Primary-pigEC; RW.4; Primary-pig ESC; 52-013; OVCAR-5; S5Y5; Primary-h Bone OC-like; SA87; INT-407; SAV-I; Primary-pig Fetus Hep; SCC-68; P69; HNPSV-1; CaSki; SK-CO15; Primary-pig Iliac EC; SK-N-DZ; Hep2; SKOV31p.1; Primary-pig Mandible Ameloblast; SNB 19; Primary-cow Joint Synovial; Primary-h Fetus FB; Primary-pig Mandible Odontoblast; SW1353; Primary-pig NP Neuronal; SW948; Primary-pig Oral MucosaEpiC; CRL-2102; Primary-pig PancreasIslets; T4-2; Primary-pig PulmonarySMC; TE-85; Primary-pig Salivary Acinar; THP-1; Primary-pig SynoviumSC; BME-UV1; KG-1; D4T; HUES-9; Primary-mouse Hippocampus Neuronal; ECV304; NRK; Primary-mouse Kidney Mesangial; D407; 10T1/2 cell line; and Primary-h Foreskin Melanocyte.

With reference to FIGS. 4, the sample can be introduced into the microfluidic device (504). Prior to loading the sample(s) into the microfluidic device, however, the device can be incubated with a solution to prevent small molecule absorption into the surface of the flow layer. For example, the microfluidic device can be incubated with a solution of BSA 1 mg mL⁻¹ in PBS at room temperature for at least 30 min.

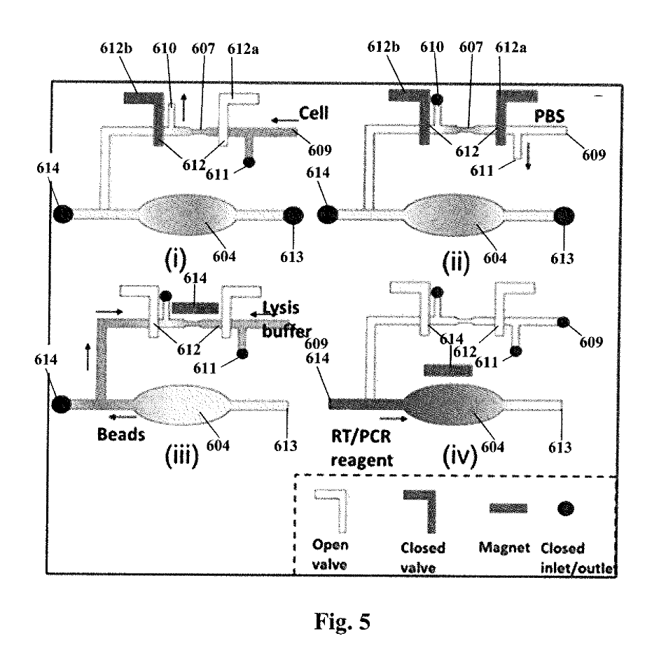

With reference to FIG. 5, the sample can be added to the microfluidic device through the cell inlet (609). In certain embodiments, the cell suspension can be added to the microfluidic device by, for example, a syringe pump, a pipette, or a tube connected to a cell harvester.

The control valves (612) can be used to manipulate the direction of the flow. The control valves can be closed by increasing the pressure in the control valve thereby precluding movement from the channels in the flow layer. For example, at atmospheric pressure the valves will be open. If the pressure is increased to approximately 10 psi the valves will be closed. In certain embodiments, the pressure to close the valves is about 5 psi to about 20 psi. In certain embodiments, the pressure to close the valves can be at least about 4 psi, at least about 6 psi, at least about 8 psi, at least about 10 psi, at least about 12 psi, at least about 14 psi, at least about 16 psi, at least about 18 psi, or at least about 20 psi. In certain embodiments, the pressure to close the valves is from about 6 psi to about 12 psi. While the sample is added to the microfluidic device, the downstream control valve (612b) can be closed and all outlets (611, 614, 615) can be closed with a plug except the cell trapping outlet (610) so that the cell suspension travels through the cell trap (607) and out of the microfluidic device through the cell trapping outlet (610).

With respect to the array, the multiplexing valves (e.g., 312c) can control which analysis unit (e.g., 301) traps the single cell. Each analysis unit has its own multiplexing valve (e.g. 312c) that controls whether the unit is open or not. For example, if only one analysis unit in an array with six analysis units is to trap a cell, the multiplexing valves (e.g. 312c) of the other five analysis units close the channels leading to the five cell trapping units (e.g., 305) not to be used. Likewise, multiplexing valves (e.g. 312c) in an array can prevent the beads and reagents from reaching certain cell trapping units (e.g., 305) and reaction chambers (e.g., 304).

With reference again to FIGS. 4, a single cell can be trapped (506). The cell trapping unit can be observed under a microscope to determine if the cells are flowing through the cell trapping unit and whether a cell has been trapped in the cell trap. To ensure a cell is trapped, narrowing of the channel (e.g., 108, 308) should make the width of the channel of the cell trapping unit smaller than the average width of the cell type to be trapped.

Once a single cell is trapped, excess cells can be removed from the microfluidic device (508). For example in FIG. 5, the upstream control valve (612a) can be closed while the cell washing outlet (611) can be opened to direct (the potentially cell-containing) carrier fluid out through the cell washing outlet (611). Additional buffer can be added to the device through the cell inlet (609) to ensure all non-trapped cells are removed from the microfluidic device through the cell washing outlet (611). In certain embodiments, the flow can still be maintained after a cell is trapped. In certain embodiments, the flow will wash out any upstream cells.

The trapped cell can be chemically lysed (510) by adding a lysis solution through the cell inlet (609). Any lysis buffer known to one of ordinary skill in the art can also be used. For example, the lysis solution can be made of 100 mM TrisHCl, pH 7.5, 500 mM LiCl, 10 mM EDTA, 1% LiDS, 5 mM dithiothreitol. Alternatively, the cells can be physically lysed. For example, the cells can be lysed using ultrasound (high-frequency energy). During the cell lysis procedure, both the upstream (612a) and downstream (612b) valves are open, while the cell trapping outlet (610), cell washing outlet (611), and bead/reagent inlet (614) are plugged shut. This allows the lysis buffer to travel to the trapped cell.

With reference to FIGS. 1B, 4, and 5, genetic material from the lysed cell can be captured with microbeads (512). In certain embodiments, the microbeads can be magnetic and can travel to the lysed cell via an external magnet (614). In certain embodiments, the microbead is a superparamagnetic particle with a polymer shell. In certain embodiments, the microbeads are functionalized with a primer (201) to capture the genetic material. In certain embodiments, the primer (201) can be specifically designed to capture (i.e., anneal to) mRNA (202). The primer can be, for example but not limited to, an oligo-dT designed to capture polyadenylated mRNA (e.g., Oligo(dT)₂₅). The principle of mRNA capture relies on base pairing between the poly A tails of the mRNA and the oligo(dT) residues covalently coupled to the surface of the beads. Nucleic acids can be covalently attached to microbeads with any of several methods known to those of skill in the art. For example, an carboxylic or amino group can be incorporated onto the 5′ or 3′ end of an oligonucleotide or PCR primer and reactive groups (e.g., —COOH carboxylic acid; —RNH2 primary aliphatic amine; —ArNH2 aromatic amine; —ArCH2Cl chloromethyl (vinyl benzyl chloride); —CONH2 amide; —CONHNH2 hydrazide; —CHO aldehyde; —OH hydroxyl; —SH thiol; —COC— epoxy, etc . . . ) can be incorporated on the microbeads for covalent attachment. By way of example, but not limitation, amine-modified oligos can then be reacted with carboxylate-modified micro-spheres with carbodiimide (e.g., EDAC) chemistry in a one-step process at pH 6-8.

By mixing the functionalized beads with the cell lysate, mRNA templates from a single cell can be captured and purified on the surface of the beads.

Once the mRNA (202) is captured, the microbeads with the captured mRNA can be transported to the reaction chamber (514) to undergo reverse transcription (516) to form a cDNA template (203). For example, if the microbeads are magnetic, they can travel from the cell trap (607) to the reaction chamber (604) via an external magnet (614). In certain embodiments, once the magnetic microbeads reach the reaction chamber (604) the device can be placed on a magnet to hold the microbeads within the reaction chamber. With the cell trapping outlet (610) and cell washing outlet (611) closed and the upstream (612a) and downstream (612b) valves open reverse transcription reagents can be added into the microfluidic device reaction chamber (604) via the bead/reagent inlet (614) or in the case of the array the cell inlet (e.g. 309), followed by the closure of all the inlets and outlets. Next, reverse transcription can take place. For example, a pulsed temperature reverse transcription protocol can be carried out to create cDNA templates (203).

The cDNA templates can then be amplified. For example, PCR reagents can be introduced into the microfluidic device which can simultaneously flush away the reverse transcription reagents. In certain embodiments, the external magnet holds the magnetic beads stationary during the influx of PCR reagents. Once the PCR reagents completely fill the reaction chamber, all the inlets and outlets can be sealed with plugs. At this point, the microdevice can be placed on the stage of a fluorescent microscope to analyze gene expression (518). For example, quantitate the amplified product (204) can be quantified with real-time PCR (516) using a primer/probe set (205) and a detection reagent (206).

In certain embodiments, for reverse transcription (RT) the bead-bound oligo(dT) can function as a primer for the synthesis of cDNA. In certain embodiments, after RT, the synthesized cDNA templates can be amplified while the accumulation of products can be real-time quantified using a hydrolysis probe/primer set (e.g., TaqMan®) (FIG. 1B). In certain embodiments, the reagent probe/primer can include a fluorescein amidite (FAM) reporter dye, a minor groove binder (MGB) and a nonfluorescent quencher (NFQ). When the probe is intact, by the Forster resonance energy transfer, the reporter fluorescence is suppressed by the quencher. During a PCR annealing process, the probe will bind to a complementary region of the target template. The quencher will be cleaved from the probe during the subsequent elongation process causing fluorescence of the reporter dye to increase. In certain embodiments, a typical RT reaction of 10 min at 25° C. and 50 min at 42° C. can be used.

In certain embodiments, to correct for fluorescent fluctuations due to batch-to-batch changes in cavity volume and PCR component concentrations, a passive reference (e.g., ROX) can be employed to normalize the FAM signal during real-time measurements. In certain embodiments, fluorescent images of the beads were taken in two different colors (e.g., ROX and FAM) after each PCR cycle.

PCR can be conducted by any commonly understood method. In certain embodiments, each PCR process can be initialized and thermocycled with the following protocols: 10 min at 95° C., followed by 35 cycles of 15 s at 95° C. and 1 min at 60° C.

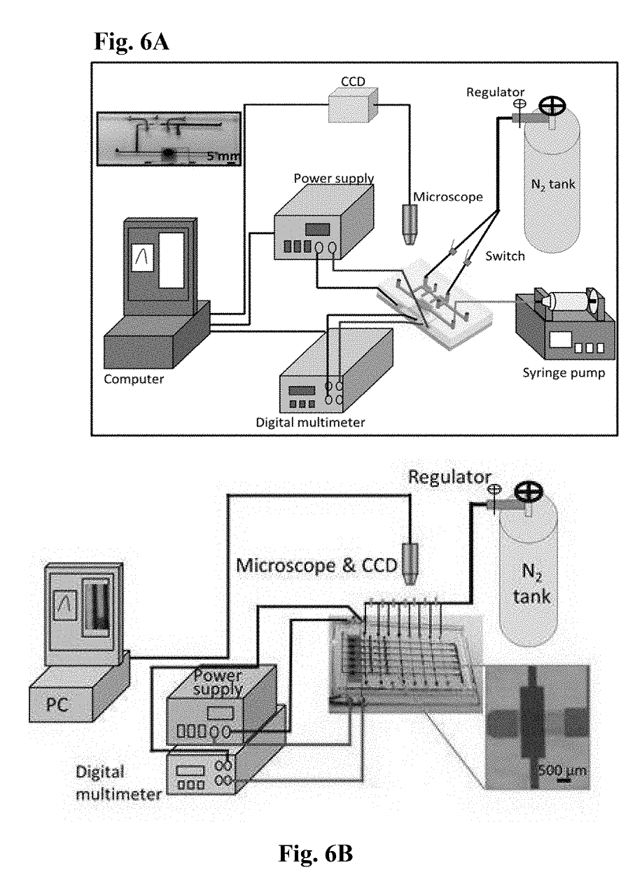

FIG. 6 presents two examples of an example setup of certain embodiments of the disclosed subject matter. For example, FIG. 6A represents an exemplary setup for a single unit microfluidic device, and FIG. 6B represents an example setup for a microfluidic array. In certain embodiments, closed-loop temperature control of the device chambers can be achieved using an integrated temperature sensor and heater. In certain embodiments, a computer controls the temperature. In certain embodiments, the temperature sensor and heater can be controlled with an algorithm In certain embodiments, the heater can be connected to a power supply (e.g., DC). In certain embodiments, the microfluidic valves of the device can be controlled by individual regulators (e.g., pressure regulators). In certain embodiments, the fluorescent intensity of the reaction in the reaction camber can be measured with a microscope. In certain embodiments, the microscope is an inverted epifluorescence microscope.

Additional aspects and embodiments of the disclosed subject matter are illustrated in the following examples, which are provided for better understanding of the disclosed subject matter and not limitation.

### EXAMPLE 1

### A Single-Cell Microfluidic Device

Illustrated herein is a microfluidic device that can allow RT-PCR analysis of single cells (FIG. 1A).

The device was capable of cell-trapping, cell lysis and bead-based RT-qPCR in a single unit. Hydrodynamic forces were employed with the device for efficient single-cell isolation and immobilization. Once immobilized, single cells were lysed chemically and mRNA templates from the lysate were captured using microbeads.

### Microfluidic Device Design

The device consisted of a temperature control chip with an integrated heater and temperature sensor, a polydimethylsiloxane (PDMS) microchamber, and a cell trapping unit (FIG. 1A). First, a single elliptically shaped reaction chamber (7.7 mm in length, 5.7 mm in width, 15 μm in height and 658±25 nL in volume) was designed for the two-step RT-qPCR process. The cell trapping unit consisted of a neck-shaped channel (800 μm in length, 100 μm in width and 15 μm in height) with a protruding structure (i.e., the portion of the mold that narrows the channel) that reduced the channel width from 100 μm to 5 μm. The cell trapping unit was also equipped with a cell tapping flow outlet, a cell washing outlet, and two pneumatic control channels (600 μm in length, 400 μm in width and 80 μm in height) to divert flow for cell trapping and lysis. A serpentine-shaped temperature sensor (linewidth: 50 μm) and heater (linewidth: 400 μm) were integrated beneath the center of the reaction chamber. In addition, to inhibit reagent evaporation and diffusion caused by PDMS porosity, a transparent and pressure sensitive adhesive film (3 mm in length and 0.5 mm in width) was bonded on top of the reaction chamber.

### Microfluidic Device Fabrication

An illustration of the fabrication process is shown in FIG. 3.

In general, the microfluidic device was fabricated using multi-layer soft lithography microfabrication techniques. Chrome (10 nm) and gold (100 nm) thin films were deposited and patterned onto a glass slide (Fisher HealthCare, Houston, Tex.) followed by passivation. AZ 4620 photoresist (Clariant Corp., Branchburg, N.J.) was first spun coated and patterned. Once developed, the photoresist was heated up to 200° C. for 1 h, which is above the glass transition temperature of the photoresist. Thus, the reflowing of the photoresist formed channels with a rounded cross section. Then, on the same wafer, SU-8 photoresist (MicroChem Corp., Newton, Mass.) was spun-coated and patterned to define the other parts of the flow layer mold. In parallel, the mold for the control layer was fabricated from SU-8 and measured using a Dektak 3 profilometer. Then, PDMS (Dow Corning) was poured over the molds and an additional evaporation barrier was embedded in the flow layer PDMS. Sheets bearing the microfluidic features were then peeled off the mold followed by inlet and outlet hole punching. Also, uncured PDMS was spun on a wafer to form a featureless membrane (20 μm in thickness). The membrane was then sandwiched between the flow and control layer by oxygen plasma. Finally, the PDMS device was bonded to the heater and sensor by oxygen plasma resulting in a packaged device. The holes in the flow layer were punched with a 1.5 mm diameter mechanical punch (Harris Uni-Core punch). The thickness of the PDMS was 3±0.1 mm for the flow layer and 240±10 μm for the control layer.

In more detail, for the passivation process, the glass slide bearing the heater and sensor was passivated by sequentially spin coating and curing a 10 μm layer of SU-8 photoresist (MicroChem SU-2010 3500 rpm for 45 seconds, 95° C. for 10 min for curing the photoresist), followed by a 10 μm of PDMS (Dow Corning PDMS 5000 rpm for 1 min, 80° C. for 20 min for curing PDMS). Oxygen plasma was employed to bind the passivation layer with the PDMS microfluidic device. After each RT-qPCR use, the PDMS microfluidic device was peeled off while the heater and sensor were reused.

For the SU-8 photolithography process, the SU-8 mold features were 15 μm high for the flow layer and 80 μm high for the control layer. First, at room temperature, permanent epoxy negative photoresist SU-8 was spin-coated on a cleaned 4-in silicon substrate at a speed of 3200 rpm for 45-60 seconds with an acceleration of 300 rpm/second. Then, the coated SU-8 photoresist was placed on a level hotplate for 10-15 min at 95° C. Next, the baked photoresist was exposed under UV light at a dose of 130-150 mJ/cm2 using a mask aligner (Süss MicroTec MA6 Mask Aligner). After exposure, the patterned SU-8 was placed on a hotplate for 4 minutes at 95° C. Then, the exposed SU-8 2015 photoresist was sprayed with MicroChem's SU-8 developer for 2-3 min. At the end of the development, the exposed photoresist was sprayed and washed with fresh SU-8 developer for approximately 10 seconds, followed by a second spray/wash with Isopropyl Alcohol (IPA) for another 10 seconds. The mold was dried with pressurized nitrogen. Finally, the silicon substrate bearing the SU-8 features was baked (150-250° C.) for 5 to 30 min to ensure that SU-8 properties do not change with thermal cycling.

An 80 μm high mold was built using SU-8 2075 photoresist.

For the evaporation barrier implantation, the evaporation barrier (optical adhesive film) was composed of polypropylene and designed for creating a secure seal across a microplate to prevent evaporation. The thickness of the film was 0.1 mm as measured by a vernier caliper. A two-step PDMS casting process was employed to embed the evaporation barrier above the reaction chamber. Initially, base and agent of PDMS were mixed in a 10:1 ratio. The mixture was degassed for 45 minutes and then was spin-coated on the mold at a speed of 4000 rpm for 45 second with an acceleration of 300 rpm/second followed by baking at 72° C. for 15 minutes. Next, a piece of adhesive film was stamped on PDMS at the region of the reaction chamber. After that, 15 mL uncured PDMS (10:1) was poured on the solid-state PDMS layer and baked to finalize the barrier implantation.

For the Reactive Ion Etching (RIE) bonding process, the mechanism was related to the breaking of bonds on each surface of PDMS during treatment followed by the formation of Si—O—Si bonds when the two surfaces were brought into contact. The PDMS layers or substrates were first installed in the process chamber of the Technics Series 800 RIE (Oxygen Plasma Asher). The samples were treated with oxygen plasma at 250 mTorr pressure by a power of 50 Watt for 4 seconds.

### Example Set-Up

Cells (e.g. MCF-7 cells) were incubated with media (e.g. MEM) supplemented with 10% FBS and 1% P/S, and were kept at 37° C. in a humidified incubator containing 5% CO₂. Before each trial, cells were collected through centrifugation and resuspended at 10⁸ cells per mL in the media and then kept on ice.

Prior to each on-chip test, the device was incubated with 1 mg mL⁻¹ BSA solution in PBS at room temperature for at least 30 min to prevent small molecule absorption into the PDMS surface.

Closed-loop temperature control of the device chambers was achieved using the integrated temperature sensor and heater with a proportional-integral-derivative (PID) algorithm implemented in a LabVIEW (National Instruments Corp., Tex.) program on a personal computer. The resistance of the sensor was measured by a digital multimeter (34420A, Agilent Technologies Inc., Calif.), and the heater was connected to a DC power supply (E3631, Agilent Technologies Inc., Calif.). The microfluidic valves of the device were controlled by individual pressure regulators (Concoa, Virginia Beach, Va.) interfaced via 20 gauge stainless steel tubing (BD, Franklin Lakes, N.J.) and Tygon tubing (ID: 0.79 mm, OD: 2.38 mm, Saint-Gobain, Grand Island, N.Y.).

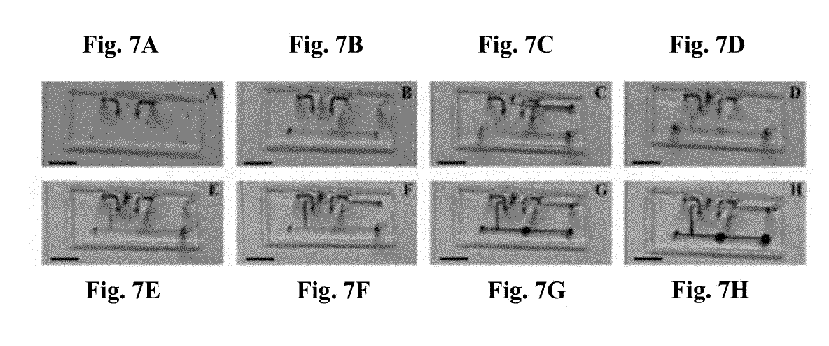

The inlets and outlets of the device were sealed off by polycarbonate plugs. The fluorescent intensity of the reaction was measured from images acquired by an inverted epifluorescence microscope (IX81, Olympus, Center Valley, Pa.) with a CCD camera (c8484, Hamamatsu, Boston, Mass.) of the reaction chamber. The schematic of the example set-up is shown in FIG. 6. FIG. 7 demonstrates the experimental procedures, wherein: (A) Before fluid was introduced into the chamber; (B) Bead introduction; (C) Cell trapping; (D) Cell washing; (E) Bead and cell mixing; (F) Cell lysis; (G) RT reagent introduction; and (H) PCR reagent introduction. Scale bar: 1 cm. These processes are disclosed in greater detail below and in the following examples.

### Bead Preparation

Before introduction into the device, the beads were washed using binding buffer (20 mM Tris-HCl, pH 7.5, 1.0 M LiCl, 2 mM EDTA) from the Dynabeads® mRNA Kit and resuspended in binding buffer in a vial using a 30 s vortex. A specific volume of beads were transferred to an RNase-free tube and suspended in binding buffer for 10 seconds. The tube was placed over a magnet for 1 min, the supernatant was discarded and the binding buffer was added to resuspend the beads. Following the final supernatant removal, the beads were suspended in 2 μL binding buffer (approximately 7.5×10⁶) and introduced to the device using a microcapillary pipette.

The beads entering the chamber were retained by an external magnetic placed underneath the chip, and the approximate number of beads was determined by analysis of the microscope image using ImageJ.

### On-Chip Cell Processing

During the on-chip cell processing, the cell washing outlet was sealed using a plastic plug, the downstream valve to cut off cell carrier flow to the chamber was closed, and the upstream valve and the cell trapping outlet was opened. The loading cell concentration was diluted and introduced cells to the chip using a syringe pump. Once a single cell was immobilized at the trap because it is unable to pass through the narrowing in the cell trap, the upstream valve was closed and the cell washing outlet was opened, directing subsequent cells away from the trap. Then, the cell trapping outlet was sealed and the upstream valve was opened to introduce lysis buffer through the cell inlet. Meanwhile, the downstream valve was opened and the beads were moved to the lysate using a magnet. The beads and lysate were mixed by magnetic motion for 10 min to capture the released mRNA onto the beads' surface. The beads with bound mRNA were then moved to the reaction chamber and extracellular RNA and debris were removed by buffer washing. The magnet was used to move the beads to the lysate thereby capturing all mRNA. Then buffer was infused into the chamber while the magnet held the beads stationary. This buffer flushed out lysate products from the device.

### Integrated On-Chip RT-qPCR

Each RT was performed in the device using the TaqMan® reverse transcription reagents. RT reagent was first pipetted into the device while the beads were immobilized in the chamber by placing the device on a magnet. Meanwhile, the cell trapping and washing outlets were closed and the upstream and downstream valves were opened. Once the reaction chamber was fully filled with RT reagents, all the inlets and outlets were closed. Then a typical pulsed temperature RT protocol was carried out (e.g., 10 min at 25° C. and 50 min at 42° C.). The TaqMan® reverse transcription reagents kit included 1.5 mM magnesium chloride. The proportion of each reagent was consistent with the manufacturer's protocol.

### Quantitative Real-Time PCR

The PCR reagent was prepared with TaqMan® Gene Expression master mix and template specific primer. The PCR reagent was introduced to the device which simultaneously flushed away the RT reagent while the chip was situated on a magnet to immobilize the beads. After PCR reagent completely filled the reaction chamber, all the outlets and inlets were sealed with plugs. Then the platform was placed on the stage of a fluorescent microscope. Each PCR process was initialized and thermocycled with the following protocols: 10 min at 95° C., followed by 40 cycles of 15 sec at 95° C. and 1 min at 60° C. Fluorescent images of the beads were taken in two different colors (one reference dye and one reporter dye) after each PCR cycle.

### EXAMPLE 2

### On-chip Heater Calibration and Thermal Characterization

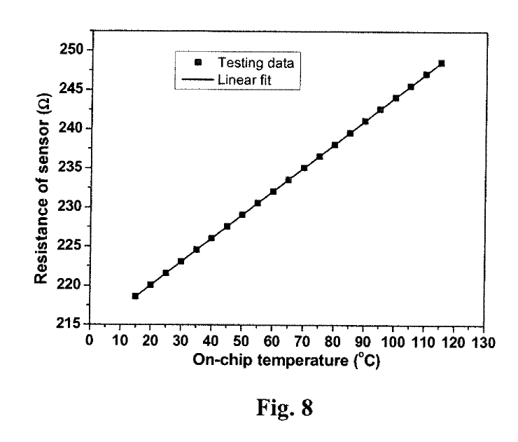

The temperature sensor was characterized to enable accurate on-chip temperature control. The chip of Example 1 was placed in a temperature-controlled environmental chamber (9023, Delta Design Inc., Calif.). Using platinum resistance temperature detector probes (Hart Scientific 5628), the temperature of the chamber was measured and the corresponding on-chip resistance was measured by a digital multimeter (Agilent 34420A). The measured resistance (R) of the gold temperature sensor was observed to vary linearly with temperature (T); thus, it exhibited a highly linear dependence on temperature. The dependence could be represented by the relationship R=R₀[1+α(T−T₀)], where R₀ is the sensor resistance at a reference temperature T₀, and α is the temperature coefficient of resistance (TCR) of the sensor (FIG. 8). The solid line represents a linear fit to the data with a regression equation: R=R0[1+1.36×10⁻³(T−T0)], (coefficient of determination R²=0.998). Fitting this relationship to the measurement data determined the values of the parameters, which were used to determine the chamber temperature from the measured sensor resistance during single-cell RT-qPCR testing. The temperature sensor had a measured resistance of 217.2Ω at a reference temperature of 14.9° C. with a TCR of 1.363×10^{-3 1}/° C., as shown in FIG. 9. Based on this, the accuracy and precision of the system was evaluated over the course of RT and 35 consecutive cycles PCR. The accuracy was computed as the difference between the set point and measured average temperature. The precision was defined as the average of the measured standard deviation of temperature variation at set point. For the RT step, with set points of 25° C. and 42° C., the temperature accuracy of 0.11° C. and 0.16° C. and the precision was 0.08° C. and 0.1° C. was measured. In parallel, for the PCR step, the accuracy of the two set points (denaturing at 95° C., annealing/extension at 60° C.) was 0.53 and 0.21 and the precision was 0.16° C. and 0.14° C. The chip achieved target temperatures with minimal overshoot (<10 s). All these results indicate that the chamber temperature can be controlled to produce accurate and rapid amplification reactions. The time course of temperature during this control test is shown in the FIG. 9. These results demonstrate that a typical RT (10 min at 25° C. and 50 min at 42° C.) and a 35-cycle PCR (15 sec at 95° C. and 1 min at 60° C.) processes can be fully integrated in the platform.

### EXAMPLE 3

### On-Chip Validation

A hydrolysis primer/probe set (CDKN1A primer/probe set (product number: Hs99999142_m1) and 2×10⁴ copies XenoRNA (10⁵ copies per μL), were used to demonstrate the feasibility of on-chip RT-PCR. The chip from Example 1 was used.

XenoRNA templates were reverse transcribed and amplified via 35 cycles of PCR. The amplification was compared with the no-template control (NTC). The protocol is shown in Table 1.

TABLE 1
On-Chip Validation Protocol
Protocol 1: Two-step RT-qPCR for validation testing
Pipette 0.2 μL XenoRNA to
0.5 μL bead pre-incubated chip and mix them for 10 min

| Reaction component | Volume (μL) |
|---|---|
| MgCl₂ (25 mM each) | 0.22 |
| dNTP (10 mM, 2.5 mM each) | 0.2 |
| RT buffer (10X) | 0.1 |
| Reverse Transcriptase (50 U/μL) | 0.025 |
| RNase inhibitor (20 U/μL) | 0.02 |
| RNase free water | 0.435 |
| Total | 1.0 |

Pipette the RT master mix to the chip and flush through the
reaction chamber while immobilize bead/mRNA by a magnet.
Place the chip on the heating stage and follow the conditions:

| | |
|---|---|
| 25° C. | 10 min |
| 42° C. | 50 min |

Place the chip on ice and pipette PCR reagents to the chip:

| | |
|---|---|
| Reaction component | Volume (μL) |
| PCR master mix (2X) | 0.5 |
| XenoRNA assay (20X) | 0.05 |
| PCR grade water | 0.45 |
| Total | 1 |

Place the chip on the heating stage
and follow the thermal cycling conditions:

| | UDG | Enzyme | PCR | |
|---|---|---|---|---|
| | Incubation | activation | Cycle (35 cycles) | |
| Step | Hold | Hold | Denature | Anneal/Extend |
| Temperature | 50° C. | 95° C. | 95° C. | 60° C. |
| Time | 2 min | 10 min | 15 s | 1 min |

Test the fluorescent intensity of the PCR products at the end of PCR.

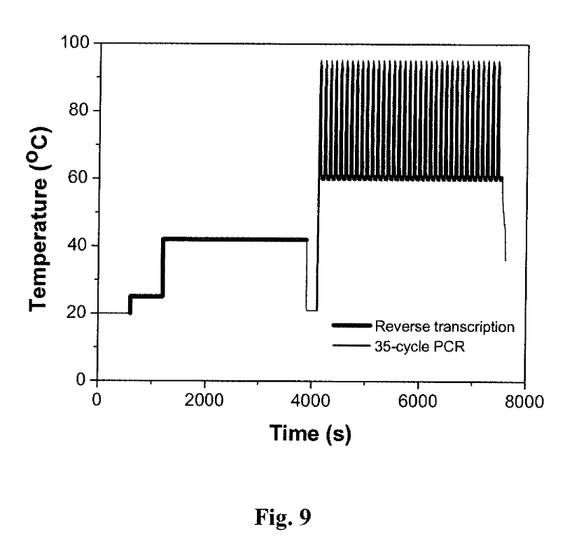

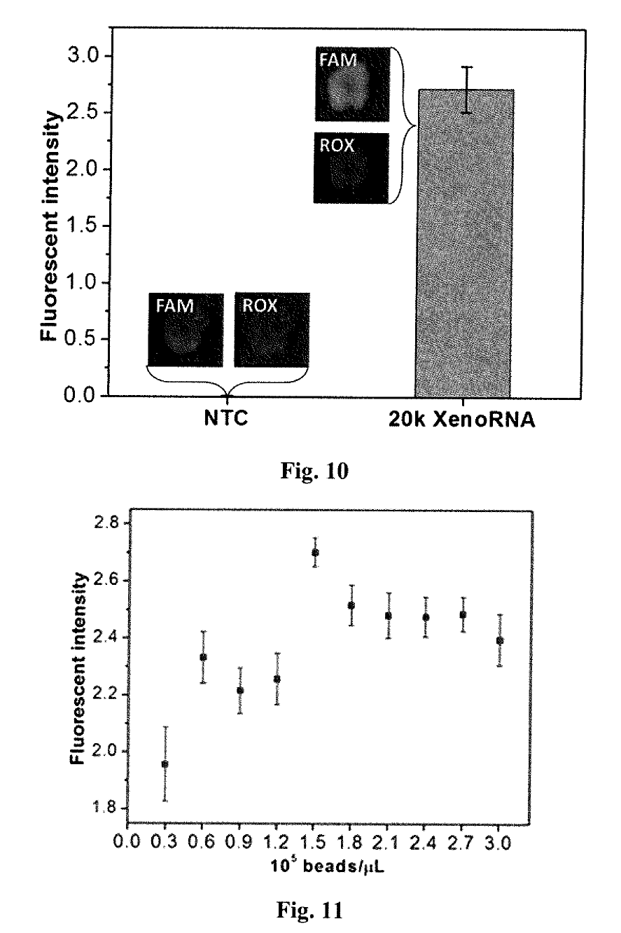

The fluorescent images and background subtracted fluorescent intensity are shown in FIG. 10 (error bar was obtained from a triplicate). For the on-chip RT-PCR of 2×10⁴ copies XenoRNA, the fluorescent image of reporter showed much greater fluorescent intensity than the NTC sample. The mean fluorescent intensity value of three XenoRNA samples after 35-cycles of PCR was 2.7±0.2 compared to 0±0.05 with the NTC. This indicated there was a significant amplification of XenoRNA templates and negligible amplification of the NTC. Furthermore the consistent fluorescent intensity indicates that the reagent concentrations were stable during on-chip RT-PCR. Thus, the reagent absorption and evaporation during the thermal cycling process were effectively inhibited.

### EXAMPLE 4

### Optimization of Bead Volume

In this example, the number of magnetic micro beads needed in the microchamber for RT-qPCR was investigated. The chip from Example 1 was used. Oligo (dT)₂₅ bead was used and was composed of a superparamagnetic particle and a polymer shell.

With the microchamber containing varying numbers (from 7.5×10⁵ to 7.5×10⁶) of beads, XenoRNA templates (10⁵ copies), approximately representing the amount of mRNA contained in a single cell, were amplified on the chip via 35 cycles of RT-PCR and detected by hydrolysis probes. The fluorescent intensity of the beads was measured at the end of the 35-cycle RT-PCR process for each bead quantity (FIG. 11 (Error bars were obtained from triplicates)). The protocol is shown in Table 2.

TABLE 2
Bead Quantity Optimization
Protocol 2: Two-step RT-PCR for bead quantity optimization

| Reaction component | Volume (μL) |
|---|---|
| XenoRNA (100,000 copies/μL) | 1.0 |
| Oligo(dT)₂₅ bead (10⁶) | 0.75 1.5 2.25 3 3.75 4.5 5.25 6 6.75 7.5 |
| MgCl₂ (25 mM) | 1.1 |
| dNTP (10 mM, 2.5 mM each) | 1.0 |
| RT buffer (10X) | 0.5 |
| Reverse Transcriptase (50 U/μL) | 0.125 |
| RNase inhibitor (20 U/μL) | 0.1 |
| RNase free water | 1.075 0.975 0.815 0.775 0.675 |
| | 0.575 0.475 0.375 0.275 0.175 |
| Total | 5.0 |

Place the tubes in the thermal cycler and follow the conditions:

| | |
|---|---|
| 25° C. | 10 min |
| 42° C. | 50 min |

Place the 5 μL-RT products on ice and pipette PCR reagents:

| | |
|---|---|
| Reaction component | Volume (μL) |
| PCR master mix (2X) | 10.0 |
| XenoRNA assay (20X) | 1.0 |
| PCR grade water | 4.0 |
| Total | 20.0 |

Place the tubes in the thermal cycler
and follow the thermal cycling conditions:

| | UDG | Enzyme | PCR | |
|---|---|---|---|---|
| | Incubation | activation | Cycle (35) cycles | |
| Step | Hold | Hold | Denature | Anneal/Extend |
| Temperature | 50° C. | 95° C. | 95° C. | 60° C. |
| Time | 2 min | 10 min | 15 s | 1 min |

Test the fluorescent intensity of the PCR products.

The fluorescence intensity, and hence the PCR reaction yield, initially increased with the number of beads in the chamber, reaching a maximum value at 3.75×10⁶ beads, and then decreased as the bead quantity further increased. According to manufacturer-supplied information on the XenoRNA capture capacity of oligo(dT)₂₅ functionalized beads, the optimum bead quantity (3.75×10⁶) is the number of beads approximately required to capture all the 10⁵ copies XenoRNA. Thus, the initial increase in the PCR reaction yield reflected more mRNA being captured on the increasing number of beads. When the bead quantity exceeded the optimal value and further increased, it is likely that no additional copies of XenoRNA were captured in the chamber, while the decreasing net reaction volume in the chamber (with 3.75×10⁶ and 7.5×10⁶ beads occupying roughly 9% and 19% of the chamber volume, respectively) caused a decrease in the reaction yield and the resulting fluorescence intensity. Thus, a bead quantity of 3.75×10⁶ can be used for reactions involving single cells, each of which was estimated to contain 10⁵ to 10⁶ copies of mRNA.

### EXAMPLE 5

### On-Chip mRNA Capture Efficiency Testing

In this example, tests were performed to assess bead-based mRNA capture efficiency. The chip from Example 1 was used.

Different copy numbers of XenoRNA samples (10⁴; 2×10⁴; 5×10⁴; and 10⁵) were captured by 3.75×10⁶ beads and the effluents were transferred to micro tubes and mixed with another bead solution including 3.75×10⁶ beads with bound oligo(dT)₂₅ primers. RT-qPCR was then performed. The protocol is shown in Table 3.

TABLE 3
Bead Quantity Optimization
Protocol 3: Two-step RT-qPCR for mRNA capture efficiency testing

| Reaction component | Volume (μL) |
|---|---|
| XenoRNA binding waste | 0.1 0.2 0.5 1.0 |
| Oligo(dT)₂₅ bead | 3.75 × 10⁶ |
| MgCl₂ (25 mM) | 1.1 |
| dNTP (10 mM, 2.5 mM each) | 1.0 |
| RT buffer (10X) | 0.5 |
| Reverse Transcriptase (50 U/μL) | 0.125 |
| RNase inhibitor (20 U/μL) | 0.1 |
| RNase free water | 1.575 1.475 1.175 0.675 |
| Total | 5.0 |

Place the tubes in the thermal cycler and follow the conditions:

| | |
|---|---|
| 25° C. | 10 min |
| 42° C. | 50 min |

Place the 5 μL-RT products on ice and pipette PCR reagents:

| | |
|---|---|
| Reaction component | Volume (μL) |
| PCR master mix (2X) | 10.0 |
| XenoRNA assay (20X) | 1.0 |
| PCR grade water | 4.0 |
| Total | 20.0 |

Transfer the reagents to the real-time PCR
system and follow the thermal cycling conditions:

| | UDG | Enzyme | PCR | |
|---|---|---|---|---|
| | Incubation | activation | Cycle (40 cycles) | |
| Step | Hold | Hold | Denature | Anneal/Extend |
| Temperature | 50° C. | 95° C. | 95° C. | 60° C. |
| Time | 2 min | 10 min | 15 s | 1 min |

Fluorescent intensity was tested
by the ABI system (HT 7900 real-time PCR)

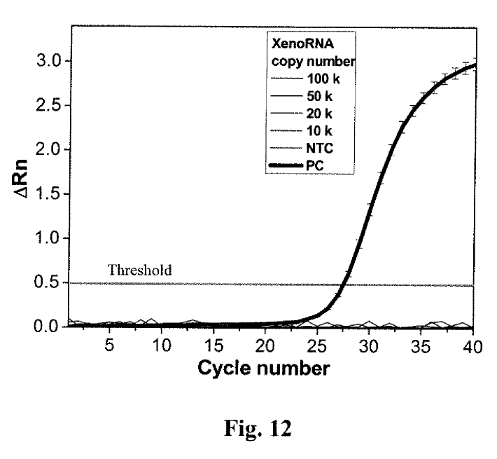

Under these conditions the same primers were used allowing for direct comparison of the binding effluent and positive control qPCR. The results are shown in FIG. 12 (Error bars were obtained from triplicates). The value of ΔRn, indicating the magnitude of the fluorescent signals and, therefore, amplification generated by PCR, was 2.9 for positive control (PC, 105 XenoRNA with 3.75×10⁶ beads) after 40 cycles of PCR (FIG. 12). While for the effluents the ΔRn values remained below the threshold. Thus, it was concluded that after bonding, all the XenoRNA were captured by 3.75×10⁶ beads and an undetectable amount of free RNA templates were residual in the binding waste. In addition, the lack of amplified products in the effluent verified that there was no significant bead loss as the chamber was flushed with buffer.

### EXAMPLE 6

### PCR Efficiency, Sensitivity And Repeatability

In this example, on-chip RT-qPCR was performed using known copies of XenoRNA and compared to in-tube bead-based and solution-phase RT-qPCR results performed under identical conditions to test the PCR efficiency, sensitivity and repeatability of the microfluidic approach. The chip from Example 1 was used. The details of the procedure are shown in Table 4.

TABLE 4
PCR Efficiency, Sensitivity and Repeatability Testing Protocol
Protocol 4: Two-step RT-qPCR for
efficiency, sensitivity and repeatability testing

| Reaction component | Volume (μL) |
|---|---|
| XenoRNA (100,000 copies/μL) | 0.1 0.2 0.5 1.0 |
| Oligo(dT)₂₅ bead | 3.75 × 10⁶ |
| MgCl₂ (25 mM) | 0.22 |
| dNTP (10 mM, 2.5 mM each) | 0.2 |
| RT buffer (10X) | 0.5 |
| Reverse Transcriptase (50 U/μL) | 0.025 |
| RNase inhibitor (20 U/μL) | 0.02 |
| RNase free water | 0.035 |
| Total | 1.0 |

Pipette the RT master mix to the chip
and flush through the reaction chamber
while immobilize bead/mRNA by a magnet.
Then perform on-chip RT follow the thermal conditions:

| | |
|---|---|
| 25° C. | 10 min |
| 42° C. | 50 min |

Prepare PCR reagents:

| | |
|---|---|
| Reaction component | Volume (μL) |
| PCR master mix (2X) | 1 |
| XenoRNA assay (20X) | 0.1 |
| PCR grade water | 0.9 |
| Total | 2.0 |

Pipette the PCR reagents to the
chip and follow the thermal cycling conditions:

| | UDG | Enzyme | PCR | |
|---|---|---|---|---|
| | Incubation | activation | Cycle (35 cycles) | |
| Step | Hold | Hold | Denature | Anneal/Extend |
| Temperature | 50° C. | 95° C. | 95° C. | 60° C. |
| Time | 2 min | 10 min | 15 s | 1 min |

Test the fluoroscent intensity of the
PCR products at the end of each cycle.
For the in-tube testing, using the same
volume, qPCR were performed by ABI system.

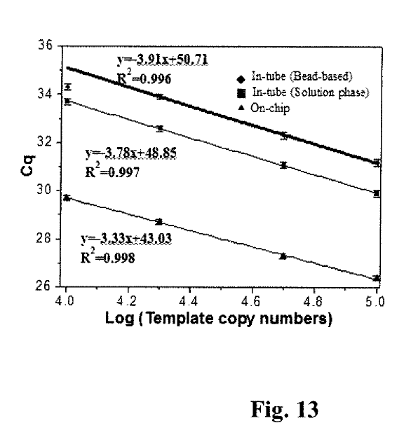

For on-chip RT-qPCR, the mean Cq values with 10⁴; 2×10⁴; 5×10⁴; and 10⁵ copies XenoRNA were 29.7; 28.7; 27.3; and 26.4 respectively. The corresponding in-tube bead-based Cq values were 34.3; 33.9; 32.3; and 31.2 and the solution-phase Cq values were 33.7, 32.6, 31.1 and 29.9 respectively. Thus, the on-chip reactions had much lower mean Cq values than in-tube reactions, suggesting a more sensitive amplification process in the microfluidic device under the given conditions. Additionally, the PCR efficiency defined by (10^{-1/k}−1)×100% was evaluated, where k is the slope of the Cq as a function of the logarithm of the template copy number (FIG. 13 (error bars were obtained from triplicates)). Under the given conditions, the PCR efficiency for the on-chip bead-based PCR testing (99.7%) was considerably higher than those for in-tube bead-based PCR (80.2%) and in-tube solution phase PCR (83.9%). This improved efficiency for on-chip PCR was likely attributable to more efficient molecular interactions in the microscale reaction environment.

For solution-phase in-tube RT-qPCR first, 10 μL of 10 mM Tris-HCl was added to the tubes containing different copy numbers of bead-bound mRNA. The tubes were incubated at 75° C. to 80° C. for 2 min, then placed on a magnet. The supernatant containing the mRNA was quickly transferred to new RNase-free tubes. Finally, solution-phase RT-qPCRs were performed using freely diffused oligo(dT) primers.

### EXAMPLE 7

### Single-Cell Isolation and Lysis

In this example, single-cell trapping efficiency was investigated. The chip from Example 1 was used.

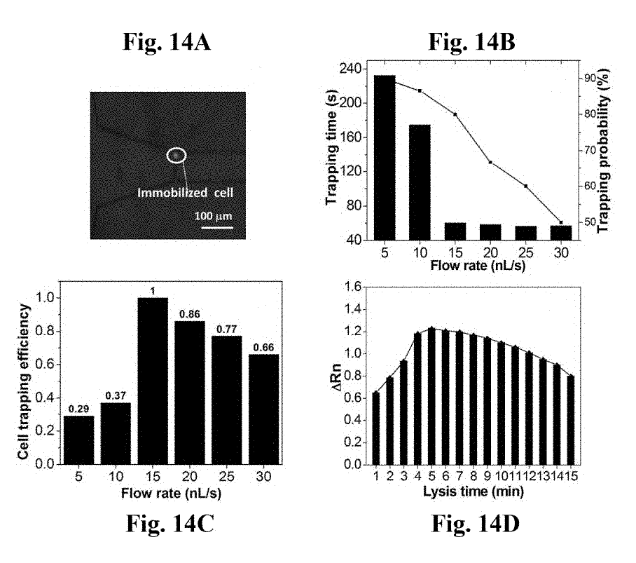

The volume ratio of Vybrant dye and cell suspension (10⁶ cells per mL) was 1:200. Using different carrier flow velocities, cells were dispensed at a fixed cell density and transported to the trapping region. The relationship between the flow rate of cell suspension and the ability of the trap to immobilize a single cell was analyzed (FIG. 14).

To assess the probability of a single cell being trapped in the device, repeated tests were conducted, in each of which a dilute cell suspension (10⁵ cells per mL) was introduced into the device via the cell input for cell trapping (FIG. 14A). The ratio of the number of tests in which a single cell was successfully trapped to the total number of tests provided a measure of cell trapping probability. Higher flow rates were found to cause a lower trapping probability as cells tended to pass through the trap because of the increased stress caused by the flow (FIG. 14B). However, a lower flow rate would require a longer trapping time, or the time from the start of cell dispensing to the instant when a single cell was trapped, which could potentially impair the cell activity. To assess the combination of these effects, a normalized trapping efficiency was defined by ε=Cp/t, where p is the trapping probability, t is the trapping time, and the scaling factor C=(t/p)max is the maximum of the ratio tip calculated from the measurements. This parameter, obtained at flow rates ranging from 5 to 30 nL s⁻¹, was found to increase with the flow rate until reaching the 100% maximum at 15 nL s⁻¹, and then decreased as the flow rate further increased (FIG. 14C). Therefore, the flow rate of 15 nL s⁻¹ can be used for cell suspensions 10⁵ cells per mL in concentration for single-cell gene expression analysis.

The effects of cell lysis time on single-cell RT-qPCR were investigated. Single cells were trapped on-chip and incubated in lysis buffer (RNase proof) for different lengths of time, while microbeads preloaded in reaction chamber were transferred to the cell trapping unit, now containing cell lysate, by movement of an external magnet. After incubation, the beads were moved back to the reaction chamber, and on-chip two-step RT-PCR was carried out to amplify the bead-bound mRNA templates and the reaction yield was analyzed (FIG. 14D). When the cells were exposed to lysis buffer for less than 5 min, the end-point fluorescent intensity of RT-PCR increased with increasing time. However, at lysis times longer than 5 minutes, the signal decreased with increasing time. For short lysis times, the mRNA release process was incomplete. Extending the lysis time can increase the amount of mRNA released and the RT efficiency until enough time has passed where all mRNA have been released. Further increasing lysis duration can cause mRNA damage by RNase as the activity of the RNase inhibitor can be affected by oxidation. Thus, 5 min can be used for cell lysis.

### EXAMPLE 8

### Fully Integrated Single-Cell Gene Expression Profiling

In this example, all steps of single-cell RT-qPCR were integrated in the device from Example 1.

Chemically induced alterations were detected in single-cell gene expression of MCF-7 cells (ATCC, Manassas, Va.) treated with methyl methanesulfonate (MMS). MCF-7 cells were incubated with MEM media supplemented with 10% FBS and 1 P/S, and were kept at 37° C. in a humidified incubator containing 5% CO₂. The Cells used herein, whether treated or not, were from the same generation of MCF-7 cells to eliminate potential generational gene expression differences.

The MCF-7 cell suspension was first diluted to 10⁵ cells per mL in a microcentrifuge tube, and mixed to homogenize the suspension and break up cell clusters. The homogenized cell suspension was then driven into the device through the cell inlet via a syringe pump while valves were used to manipulate the direction of the flow. The trapping structure was observed under a microscope. As all cells were directed through the cell trapping unit and the width of the channel at the protruding structure (i.e., the portion of the mold that narrows the channel) within the trapping unit (5 μm) is smaller than the average diameter of MCF-7 cells (18±2 μm), a single cell was immobilized in the trap.

Once a single cell was trapped, the upstream control valve (FIG. 5) was activated while the cell washing outlet was opened to direct (the potentially cell-containing) carrier fluid away from the cell trap. On average, 1.5 cells were introduced into the device per second. Single cells were consistently trapped in the microchip in 150 seconds or less. A lysis solution (100 mM TrisHCl, pH 7.5, 500 mM LiCl, 10 mM EDTA, 1% LiDS, 5 mM dithiothreitol) was used to chemically lyse the trapped cells. By mixing oligo(dT)₂₅ functionalized beads with the cell lysate, mRNA templates from a single cell were captured and purified on the surface of the beads.

The beads were then moved back to the reaction chamber and retained by an external magnet. With the cell trapping and washing outlets closed and the upstream and downstream valves open (FIG. 5), RT reagents were pipetted into the device chamber, followed by the closure of all the inlets and outlets. Then a pulsed temperature RT protocol was carried out (10 min at 25° C. and 50 min at 42° C.). Similarly, after RT, PCR reagents were introduced into the device which simultaneously flushed away the RT reagent, while the chip was situated on a magnet to immobilize the beads. Once the PCR reagents completely filled the reaction chamber, all the outlets were sealed with plugs. Then the platform was placed on the stage of a fluorescent microscope. Each PCR process was initialized and thermocycled with the following protocols: 10 min at 95° C., followed by 35 cycles of 15 s at 95° C. and 1 min at 60° C. The whole operation process is shown in FIG. 5.

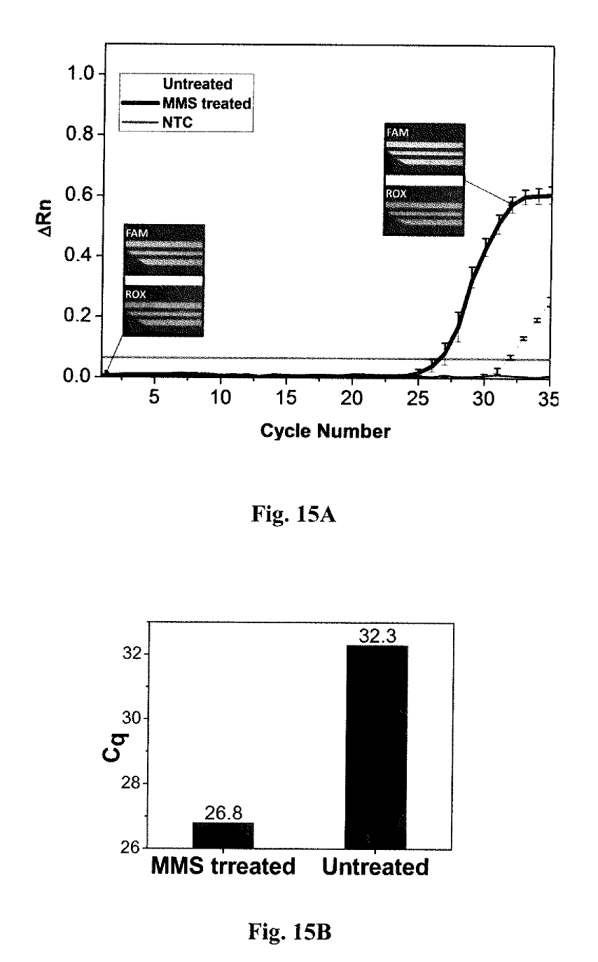

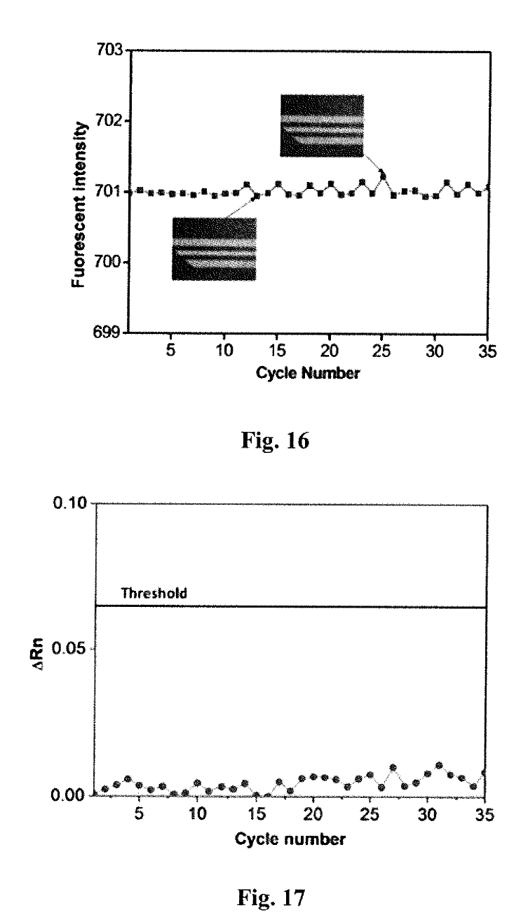

The gene expressions of single cells were assayed for the induction of CDKN1A using a hydrolysis probe/primer. The amplification of the CDKN1A gene is shown in FIG. 15A (the points and error bars correspond to mean and standard deviation of fluorescent intensity during qPCR based on five repeated tests). The threshold was calculated to be 0.07. For untreated (thin gray line) and MMS treated single cells (thick black line), the Cq values were 32.3 and 26.8, respectively (FIG. 15B). The Cq values indicate the approach was capable of detecting the MMS upregulation of CDKN1A gene expression at the single-cell level. The standard deviations of ΔRn during the whole 35-cycle qPCR were below 0.04 and 0.01 for treated and untreated single cells respectively. Furthermore, the fluorescent images of the device at the first cycle and the 35th cycle of PCR indicated there was significant amplification of the CDKN1A gene in the reaction chamber. The ROX intensity detected during the entire RT-qPCR process is presented in FIG. 16. The fluorescent intensity testing of no-template control is demonstrated in FIG. 17. The steady ROX fluorescent intensity, in addition to the constant path length during the qPCR process indicated stable reagent concentrations.

Encoding by the CDKN1A gene, which is located on chromosome 6 (6p21.2), p21/WAF1, can bind to and inhibit CDK activity, preventing phosphorylation of critical cyclin-dependent kinase substrates and blocking cell cycle progression. In the fully integrated single-cell gene expression profiling, the mean Cq value of MMS treated single MCF-7 cells was 5.48 cycles lower than the value for untreated single cells. Thus, the amount of starting templates in 2.5 h MMS treated MCF-7 cells was about 45 folds higher than in untreated single MCF-7 cells. After the MMS treatment, the transcript levels of the CDKN1A gene had been upregulated significantly and detected in the microchip. The results demonstrated the utility of the single-cell microfluidic approach for potentially enabling rapid, sensitive and reliable single-cell gene expression analysis.

### EXAMPLE 9

### Microfluidic Array For Parallelized Single-Cell Gene Expression

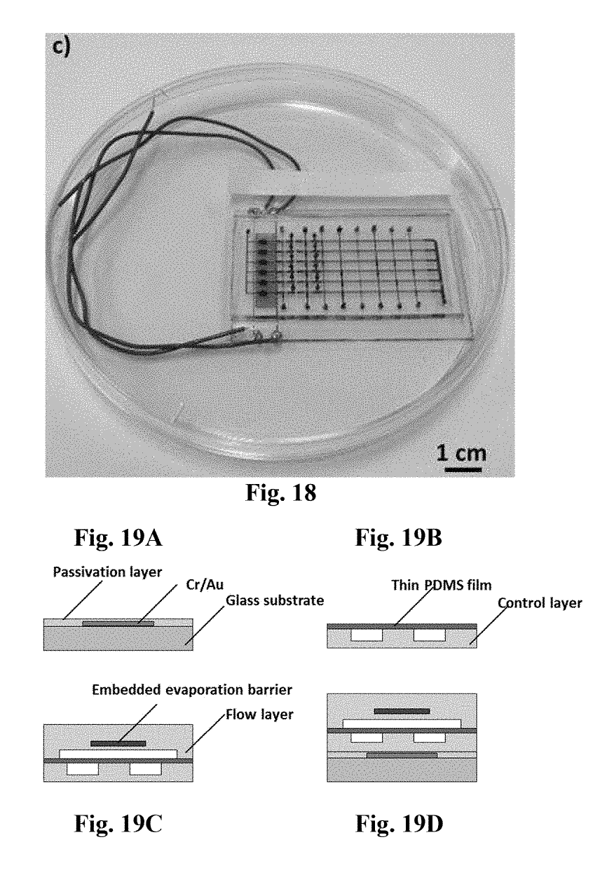

Illustrated herein is a microfluidic device that can allow RT-PCR analysis of single cells in a parallel fashion (FIGS. 2 and 18).

This device is capable of parallelized, simultaneous quantitative genetic assays of single cells, thereby providing a platform for multiplex gene detection and sequencing and allowing studies of heterogeneity in biological systems at the single-cell level.

### Microfluidic Device Design

This device consisted of six parallel analysis units connected to a single main inlet and main outlet, based on a single substrate with an integrated micro heater and temperature sensor. The analysis units were identical in design and each consisted of a cell trap, a buffer outlet, a cell outlet and an elliptically shaped reaction chamber microchamber. The cell trap was a neck-shaped flow constriction formed by a protruding microstructure (i.e., the portion of the mold that narrows the channel) in the channel between two valves (FIG. 2). The reaction chambers each have a polyethylene membrane embedded in the ceiling, which served as a barrier to minimize evaporation and associated reagent loss during thermal cycling. The integrated resistive heater and temperature sensor allowed all of the reaction chambers to be thermal cycled simultaneously. Eight individually pressurized elastomeric binary valves were arranged in a combinatorial array allowing for precise control of flow within a single analysis unit while mutually preventing flow in the other five analysis units.

### Microfluidic Device Fabrication

The fabrication process is outlined in FIG. 19.

In general, the microchip was fabricated using standard microfabrication techniques (H. Sun, T. Olsen, Q. Lin, et al. A bead-based microfluidic approach to integrated single-cell gene expression analysis by quantitative RT-PCR. RSC Advances, 2015, 5(7): 4886-4893)). Chrome (99.9%) and gold (99.99%) were deposited and patterned onto a glass substrate. AZ 4620 and SU-8 photoresist were patterned on a wafer to form the molds of the flow layer and the control layer. An evaporation barrier was embedded on top of reaction chamber. A featureless thin film was bonded to the surface of the control layer, and together bonded with the flow layer. Finally, the PDMS device was bonded to the heater resulting in a packaged device. Prior to each test, the device was incubated in BSA for 5 minutes, and the beads are washed and are injected into the reaction microchamber.

In more detail, for the heater and sensor, chrome (−20 nm) and gold (−110 nm) were deposited and patterned onto a glass substrate to make a reusable on-chip heater and sensor. Then, the glass slide bearing the heater and sensor was passivated by sequentially spin coating and curing a 10 μm layer of SU-8 photoresist (MicroChem SU-20 I 0 3500 rpm for 45 seconds, 95° C. for 10 min for curing the photoresist), followed by a 10 μm of PDMS (Dow Coming) spun coating (5000 rpm for 1 min) and baking (80° C. for 20 min).

For the control layer, by employing SU-8 photolithography, the mold of the control layer was fabricated. SU-8 photoresist 2025 (MicroChem Corp., Newton, Mass., USA) was spin-coated on a piranha cleaned 4-in silicon substrate at a speed of 3000 rpm for 45-60 seconds with an acceleration of 300 rpm/second. Then, the coated SU-8 photoresist was placed on a level hotplate for 10-15 min at 95° C. Next, the baked photoresist was exposed under UV light at a dose of 140-160 mJ/cm² using a mask aligner (Si.iss MicroTec MA6 Mask Aligner). After the post bake (95° C. for 4 min) the exposed SU-8 photoresist was sprayed with MicroChem's SU-8 developer for 3 min. The mold was then dried with pressurized nitrogen followed by a hard bake process (150-250° C. for 30 min). The thickness of the PDMS was 240±10 μm for the control layer.

For the flow layer, AZ 4620 photoresist (Clariant Corp., Branchburg, N.J.) and SU-8 were employed to fabricate the flow layer mold. First, AZ 4620 was spun coated and patterned. Once developed, the photoresist was heated up to 200° C. for 1 h, which is above the glass transition temperature of the photoresist. Thus, the reflowing of the photoresist forms channels with a rounded cross section. Then, on the same wafer, SU-8 photoresist (MicroChem Corp., Newton, Mass.) was spun coated and patterned to define the other parts of the flow layer mold. The thickness of the mold was measured using a Dektak 3 profilometer. Then, PDMS was poured over the molds and an evaporation barrier was embedded in the flow layer PDMS. Sheets bearing the microfluidic features were then peeled off the mold followed by inlet and outlet hole punching. Also, uncured PDMS was spun on a wafer to form featureless membrane (20 μm in thickness). The membrane was then sandwiched between the flow and control layer by oxygen plasma. The thickness of the PDMS was 3±0.1 mm for the flow layer.

For the device package, the PDMS layers or substrates were installed in the process chamber of the Technics Series 800 RIE (Oxygen Plasma Asher). The samples were treated with oxygen plasma at 250 mTorr pressure by a power of 50 Watt for 4 seconds. Finally, the PDMS device was bonded to the heater and sensor resulting in a packaged device.

### Example Set-Up

The fabricated device was connected to a nitrogen tank with pressure regulator (Concoa, Virginia Beach, Va.) which controlled the microvalves and, a digital multimeter (34420A, Agilent Technologies Inc., Calif.) and DC power supply, which controlled RT-qPCR reaction chamber temperature. A syringe pump (New Era Pump Systems, Inc., Farmingdale, N.Y.) was used to introduce cells, washing buffer, lysis buffer, RT reagents, and qPCR reagents into the device. An epifluorescence microscope (IX71, Olympus, Center Valley, Pa.) was used to collect real-time data. The digital multimeter and DC power supply were connected to a personal computer to allow on-chip temperature monitoring and control through a proportional-integral-derivative (PID) algorithm implemented in a Lab VIEW (National Instruments Corp., Tex.). Each analysis unit was photographed at the completion of every PCR cycle with a CCD camera (c8484, Hamamatsu, Boston, Mass.) connected to the epifluorescence microscope. The schematic of the set-up is shown in FIG. 6B.

### Bead Preparation

The beads were prepared as described in Example 1.

### On-Chip Cell Processing

During the operation of the array chip, a single analysis unit is selected through the multiplexed valves and then 4x10⁶ magnetic beads are introduced (100 nL/s for 20 s) until all analysis units have beads with a syringe pump while an external magnet is held under the chambers. The amount of beads is determined according to the manufacturer's reported bead density and verified by pipetting beads onto a glass slide and counting the total amount of beads with ImageJ software. Next a single analysis unit is reselected with the multiplexed valves and 1.25 μL of cell-containing carrier buffer are introduced (concentration: 10⁵ cells/mL, flow rate: 15 nL/s, and infusion time: 83.3 seconds) into the microfluidic device and travel through the cell trap with the first cell to reach the trapping structure becoming trapped. The width of the constriction (5 μm) is smaller than the diameter of the cells (18±2 μm) and therefore the single cell can be immobilized in the trap.

Once a single cell becomes immobilized, the back control valve is opened to allow carrier fluid, potentially containing additional cells, to bypass the trapping region and flow directly to the outlet. To further ensure that no cells remain in the outlet well, buffer is injected into the inlet well with a syringe with a 25 gauge stainless steel needle. Lysis buffer is introduced into the cell trap region of each analysis unit at a flow rate of 2 μl/min over a 5-min period to lyse the trapped cell (Sun et al. 2015). After cell lysis the beads were mixed with the cell lysate by manually moving the magnet and captured the mRNA in the cell content, based on complimentary base pairing between the polyA tails of the mRNA and the oligo(dT)₂₅ residues covalently coupled to the bead surfaces. Potential crosstalk between different analysis units (i.e., unwanted microbead movements in chambers not active in manipulation at a given time) was prevented by closing the associated valves and placement of a second, larger magnet above the inactive chambers as an added precaution. The beads were moved into the active chamber and mixed with the sample (mRNA in cell lysate or XenoRNA) by magnet-driven motion. This process is then repeated for the remaining units of the array chip until five of the RT-qPCR chambers each contain beads with bound mRNA. The sixth chamber provides no-template control. Operation of a single unit is shown in FIG. 2a.

### Integrated On-Chip RT-qPCR

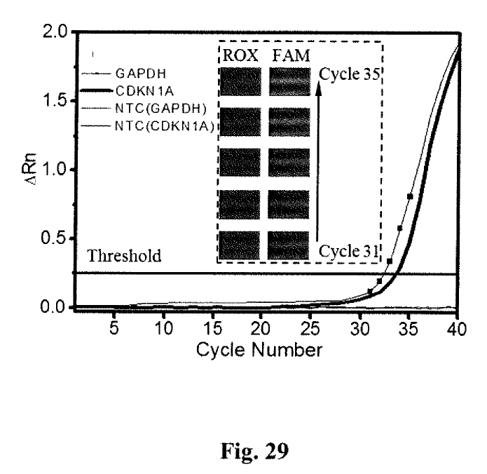

Reverse transcription is performed in all of the six chambers to convert the mRNA into cDNA, using the bead-bound oligo(dT)₂₅ as a primer and with the chamber temperatures controlled simultaneously in closed loop by the integrated heater and temperature sensor (RT: 10 min at 25° C. and 50 min at 42° C.). The resulting cDNA template for PCR is amplified (qPCR: 10 min at 95° C., followed by 40 cycles of 15 s at 95° C. and 1 min at 60° C.) while the accumulation of products is imaged on a fluorescence microscope using a hydrolysis probe/primer set (TaqMan®). The reagent probe/primer consists of a fluorescein amidite (FAM) reporter dye, a minor groove binder and a nonfluorescent quencher (NFQ). Initially, the fluorescence of FAM is suppressed by NFQ. During the PCR annealing process, the probe binds to a complementary region of the target template. Then, during the elongation process that follows, NFQ is cleaved from the probe, causing the fluorescent intensity of FAM to increase. To correct for fluorescent fluctuations due to batch-to-batch changes in cavity volume and PCR component concentrations, a passive reference dye (ROX) is employed to normalize the FAM signal during real-time measurements. Fluorescent intensities of ROX and FAM are acquired and measured at the end of each PCR cycle (FIG. 29).

### EXAMPLE 10

### On-chip Temperature Sensor Characterization For Microfluidic Array

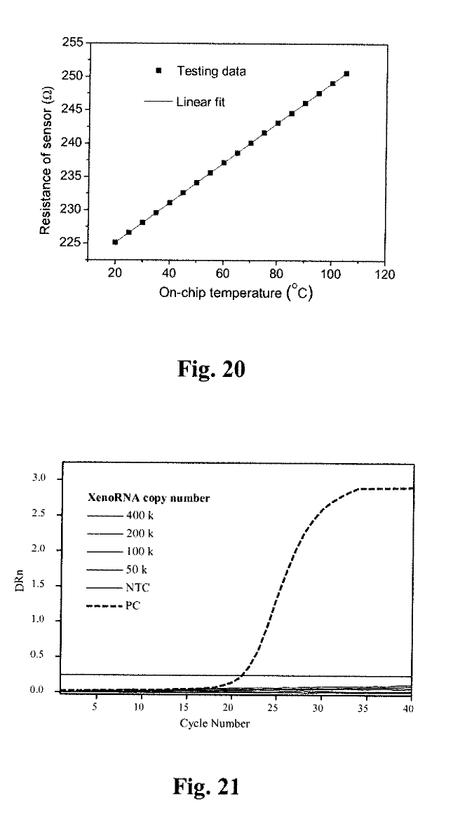

The temperature sensor was characterized to enable accurate on-chip temperature control of the microfluidic array device. The chip of Example 9 was placed in a temperature-controlled environmental chamber (9023, Delta Design Inc., Calif.). Using platinum resistance temperature detector probes (Hart Scientific 5628), the temperature of the chamber was measured and the corresponding on-chip resistance was measured by a digital multimeter (Agilent 34420A). The measured resistance (R) of the gold temperature sensor was observed to vary linearly with temperature (T). The dependence could be represented by the R=R₀[1+α(T−T₀)], where R₀ is the sensor resistance at a reference temperature T₀, and α is the temperature coefficient of resistance (TCR) of the sensor. Fitting this relationship to the measurement data determined the values of the parameters, which were used to determine the chamber temperature from the measured sensor resistance during single-cell qRT-PCR testing. The temperature sensor had a measured resistance of 225.1Ω at a reference temperature of 20° C. with a TCR of 1.318×10⁻³1/° C., as shown in FIG. 20.

### EXAMPLE 11

### On-Chip mRNA Capture Efficiency Testing For Microfluidic Array

In this example, experiments were performed to assess bead-based mRNA capture efficiency. The chip from Example 9 was used.

A bead-based approach to capture and amplify mRNA at the single-cell level was used; thus, it was first necessary to determine the capture capacity of the bead bound poly (A) tail RNA towards mRNA. XenoRNA (10⁵ copies/μL), was used to validate the sub-functions of the device. Varying amounts of XenoRNA (from 5×10⁴ to 4×10⁵ copies) was mixed with a constant quantity of poly (A) beads (4×10⁶ beads) and the residual templates was assessed in the binding effluent. This was performed in four separate arrayed analysis units of the device, where XenoRNA of varying copy number were injected and captured by the preloaded beads. The other two analysis units were used as a positive control (containing 4×10⁵ XenoRNA previously mixed with beads off-chip) and a no-template control (containing only buffer). After mixing the beads with XenoRNA for five minutes, the binding effluent was collected in each analysis unit. For the positive control and no-template control, the entire contents of each chamber (including the beads) were transferred to tubes in an RNase free environment for RT (10 min at 25° C. and 50 min at 42° C.). Finally, these RT products were analyzed by an ABT H7-7900 real-time PCR system (FIG. 21) following established protocols (10 min at 95° C., followed by 40 cycles of 15 s at 95° C. and 1 min at 60° C.). It was found the signal in these effluents and no-template control were undetectable using 4×10⁶ beads while an exponential amplification of the starting templates was observed in the PC.

Thus, it was concluded that after mixing, the XenoRNA were captured by 4×10⁶ beads and an undetectable amount of residual free RNA templates were residual in the binding waste after the 40-cycle qPCRs. In addition, the lack of amplified products in the effluent verified that there was not a significant amount of bead loss because the presence of beads in the effluent, which would have bound XenoRNA, would generate detectable amplification during qPCR.

### EXAMPLE 12

### mRNA Amplification With a Microfluidic Array

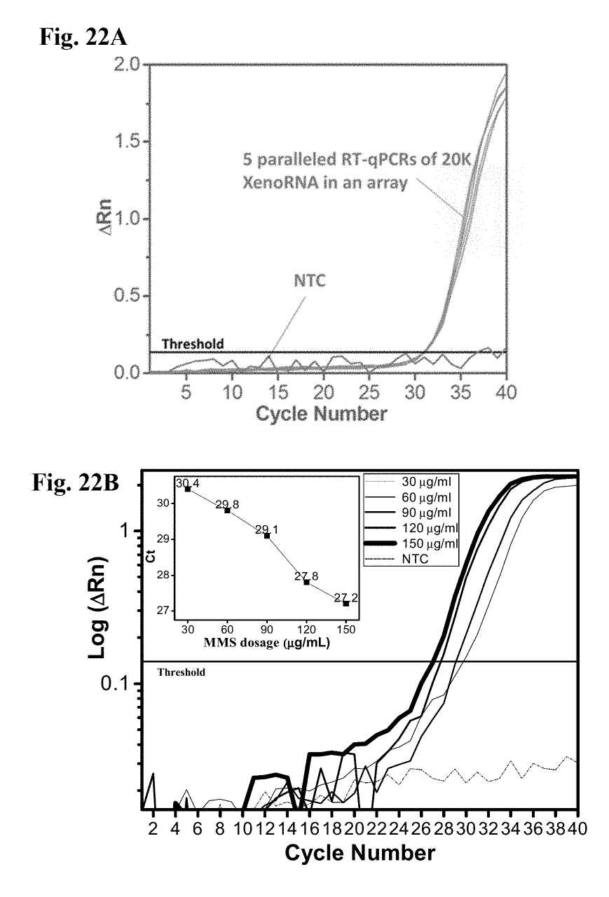

After the fluorescent microscope characterization and on-chip thermal control calibration, a fully integrated, parallelized qRT-PCR of a synthetic RNA transcript (XenoRNA) was performed in six analysis units of the chip from Example 9 to verify the consistency of genetic analysis using the arrayed microchip (FIG. 22A and B). For the five samples with 1×10⁵ copies XenoRNA, the quantification cycle (Cq) were consistent to be 31 while for the no template control (NTC), no detectable fluorescent signals were acquired. The results demonstrated that the target XenoRNA templates can be amplified by this approach and for the same starting template amount, the amplification in the separated array unit were consistent.

### EXAMPLE 13

### Validation of On-Chip RT-PCR of Model mRNA For Microfluidic Array

In this example, a similar procedure was followed as the single-cell studies except XenoRNA was injected directly into the reaction chamber through the inlet and captured onto beads, and a hydrolysis primer/probe set was used without the need for cell trapping and cell lysis. The chip from Example 9 was used.

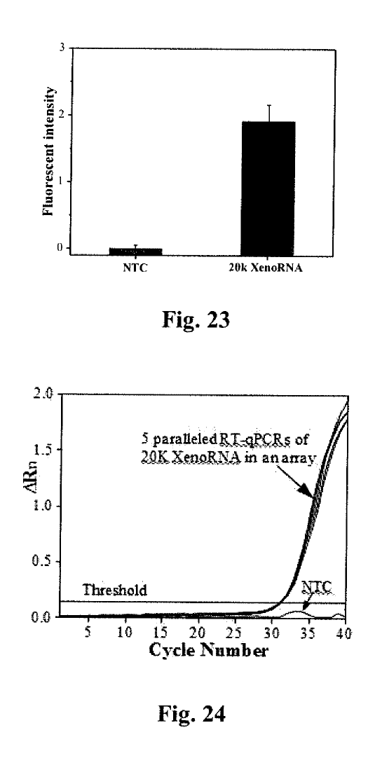

To validate the ability of the device to perform RT-PCR, the background subtracted endpoint (after 40 PCR cycles) fluorescent intensity of the reaction chamber was measured, which was compared to a no-template control. First approximately 4×10⁴ beads were injected into all analysis units and 2×10⁴ copies XenoRNA in three of the analysis units. RT and PCR reagents, without any XenoRNA templates, were introduced to the other analysis units to form no-template control analysis units. The amplification, following RT and PCR, the endpoint fluorescent intensity of the amplification was measured (background subtracted) and compared to the no-template control (FIG. 23).

For the analysis units containing XenoRNA, the fluorescent images of reporter showed much greater fluorescent intensity than the no-template control. The mean fluorescent intensity value of three XenoRNA samples following PCR was 1.9±0.26 compared to 0±0.1 with the no-template control. In contrast, there was negligible amplification in the no-template control chambers verifying that the cross contamination in the microfluidic channels was negligible when using the on-chip multiplexed microfluidic control.

On-chip RT-qPCR was validated, and the consistency of this approach was evaluated (FIG. 24) using model RNA. In five analysis units of the microchip 2×10⁴ copies of XenoRNA and 4×10⁴ beads was introduced, and RT-qPCR was performed. In the sixth analysis unit the same procedures and reagents were used except XenoRNA was not introduced. Fluorescent intensities of reaction chambers were acquired at the end of each PCR cycle.

In FIG. 24, the value of ΔRn, indicating the magnitude of the fluorescent signals and therefore amplification generated by PCR, demonstrated an exponential increase with the cycle number. Meanwhile, the quantification cycle, Cq, was set as the cycle number at which the measured fluorescence crosses a threshold of 20 σ, where σ is the standard deviation of the fluorescence intensity for the first fifteen PCR cycles. Under these conditions, the threshold value was calculated to be 0.15 and accordingly Cq was found to be 31.3 using 2×10⁴ copies XenoRNA. The results in FIG. 24 demonstrated that with the same amount of homogenized starting templates, the qPCR in all five analysis units generated significant amplification at almost the same cycle number (31.3 with a standard deviation of 0.1). Based on this, it was conclude that for the same quantity of starting templates, the amplification in the separate array units was consistent. Furthermore, the consistency of normalized fluorescent intensity (ΔRn) values during the whole qPCR process implies that the reagent concentrations were stable throughout the process. Therefore, the reagent absorption and evaporation during the thermal cycling process in the reaction chambers, which would affect the concentrations of the reagents and thus the fluorescent intensity of reporter dye (FAM), were effectively inhibited.

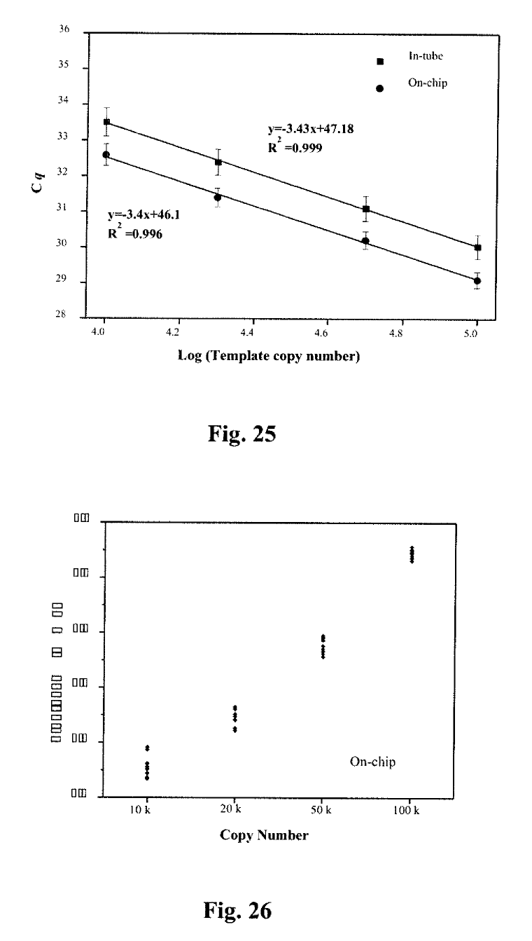

The efficiency, sensitivity and amplification yield of the nanoliter-volume scale on-chip approach was compared with the microliter-volume scale in-tube approach. XenoRNA with different copy numbers (from 10⁴ to 10⁵) were tested by in-tube and on-chip RT-qPCR respectively with results plotted and fitted linearly (FIG. 25 (mean and standard deviation are shown based on five repeated tests)).

For on-chip RT-qPCR (circles), the mean Cq values of XenoRNA with different copy numbers (from 10⁴ to 10⁵) were 32.6; 31.4; 30.2; and 29.1 with standard deviations of 0.30; 0.26; 0.24; and 0.22, while the mean Cq values for in-tube tests (squares) were 33.5; 32.4; 31.1; and 30.0 with standard deviations of 0.39, 0.36, 0.35 and 0.34. Furthermore, the PCR efficiency defined was evaluated by (10^{-1/k}−1)×100%, where k is the slope of the Cq as a function of the logarithm of the template copy number (FIG. 25). The PCR efficiency for the on-chip qPCR (96.83%) was similar to that for in-tube bead-based PCR (95.66%). Using identical starting copy numbers, the mean and standard deviation of Cq for on-chip qPCR were lower than the corresponding in-tube tests, indicating that the efficiency and sensitivity of RT-qPCR have been improved by the presented nanoliter volume reactions.

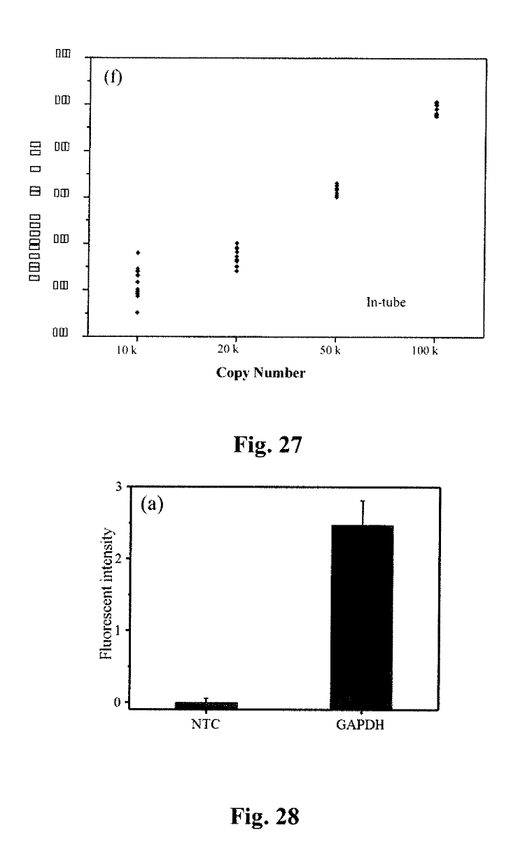

Additionally, the ΔRn value after 40 cycles of qPCR increased from 1.7 to 2.5 on-chip (FIG. 26), and from 1.6 to 2.4 (FIG. 27 (data points represent ten repeated tests) in-tube while the starting amount of XenoRNA copy numbers increased from 10⁴ to 10⁵ indicating increased PCR on-chip. The lower mean Cq values (˜1.0), the smaller Cq standard deviations (from 0.09-0.12), the increased PCR efficiency (1.17%) and ΔRn(˜1.0) of the on-chip reactions suggested improved sensitivities, less contamination, and limited degradation of samples from the smaller reaction volume, and a more integrated and automated protocol.

### EXAMPLE 14

### Measurement of Single-Cell Gene Expression With a Microfluidic Array

Integrated on-chip single-cell RT-PCR was validated with the endpoint fluorescent intensity. Three analysis units (FIG. 28) were used for cell studies while the other three chambers were used as no-template controls. Single-cell RT-PCR was performed using the procedure in Example 9.

The endpoint mean fluorescent intensity value of three single-cell samples was 2.5±0.41 compared to 0±0.1 found with the no-template control. Single MCF-7 cells were effectively lysed on-chip and the released mRNA templates were successfully captured and synthetized to cDNA. In addition, the increased standard deviation of the single-cell RT-PCR (0.41), when compared to the synthetically homogenized XenoRNA based test (0.26), indicated the variation in gene expression between cells was larger than homogenized RNA samples and suggested that the population-averaged reading of gene expression can be unable to reveal gene expression levels of individual cells.

To validate real-time amplification in the device, on-chip qPCR analysis of single cells for GAPDH and CDKN1 A expression was performed. Two microchips were prepared, one for studying GAPDH and the other for CDKNI A. Cells were introduced to the microchips following the same protocol as in Example 9. Primer/probe sets for GAPDH and CDKN I A were used. One analysis unit of each microchip was reserved as a no-template control.

FIG. 29 shows the mean ΔRn values of each test. No-template controls are also shown. The results were based on one test by an arrayed chip for each gene respectively. The FAM and ROX images acquired with the GAPDH primer/probe around the quantification cycle are also shown (inset of FIG. 29), and also demonstrates stable ROX and increasing FAM signals around the quantification cycle. The curves show exponential amplification for the targeted strands of CDKNIA and GAPDH, and the Cq difference from these curves can be used to infer differences in initial copy amounts between two genes. For GAPDH, the mean Cq value was 32.4 and for CDKN1 A, it was 33.3 which indicates that GAPDH mRNA was more abundant than CDKNIA mRNA and is consistent with and supported by existing single-cell studies.

### EXAMPLE 15

### Measurement of Drug Induced Single-cell Gene Expression With a Microfluidic Array

In this example, stress induced gene expression in single cells was investigated by treating cells with MMS, an alkylating agent, and then analyzing them by on-chip RT-qPCR. The transcript levels of CDKN1 A and the housekeeping gene GAPDH was measured in MMS treated (120 μg/mL for 2.5 h) and untreated single MCF-7 cells using the microfluidic array.

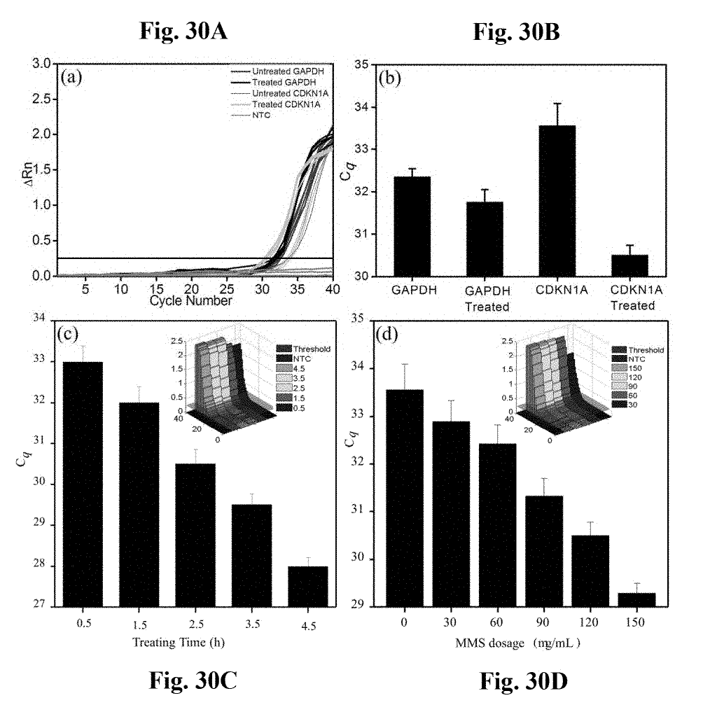

In each test, five MMS treated or untreated single cells were isolated and immobilized in five separate analysis units of the array, and the remaining unit was used as a no-template control. Similar to the above mentioned protocol, after cell trapping and lysis, the two-step RT-qPCR was initialized. The fluorescent intensity was detected and assayed using hydrolysis probe/primer sets for CDKN1 A and GAPDH respectively during the qPCR process (FIG. 30A). The mean Cq values of CDKNIA in untreated single cells was 33.5 with a standard deviation of 0.54 (FIG. 30B (each test was repeated three times and error bars represent standard deviations)). After treatment with 120 μg/mL of MMS for 2.5 h, the value decreased to 30.5 with a standard deviation of 0.23. For GAPDH in untreated single cells, the no-template control samples, no fluorescent signal was detected. The difference between the Cq of treated and untreated cells, ΔCq, was determined to be 3.0 for CDKN1 A, and for the housekeeping gene GAPDH, ΔCq was found to be approximately 0.5. Encoded by the CDKN1 A gene, which is located on chromosome 6 (6p21.2), p21/WAF1 can bind to and inhibit CDK activity, preventing phosphorylation of critical cyclin-dependent kinase substrates and blocking cell cycle progression. By treating the single cells with MMS, the transcript levels of the CDKNIA were upregulated significantly. Conversely, the transcript level of GAPDH has been found to be very consistent regardless of the cellular condition, leading to its common use as an internal control for gene expression tests. Through the above analysis, the presented approach demonstrated the ability to detect alterations in transcript levels in single mammalian cells.

The effect of drug exposure time on transcript levels of CDKN1 A in single MCF-7 cells was assayed. First, five cell suspensions were exposed to MMS for different time durations (from 0.5 to 4.5 hrs). Following exposure to MMS, the cell suspension from the five separate culture mediums were diluted and introduced to five analysis units on the microchip. Cq values of CDKN1 A RT-qPCR in MMS-treated single cells and mean 3-D qPCR amplification curves for this scenario (FIG. 30C) were plotted (each test was repeated three times and error bars represent standard deviations).

The mean Cq values decreased from 32.9 to 28.1 when the exposure time increased from 0.5 to 4.5 h. Also, the standard deviation of Cq decreased (from 0.5 to 0.2) as the exposure time increased. Furthermore, the exposure time did not have a linear relationship with CDKN1 A expression, as seen by the increased inter-unit Cq differences with increasing exposure durations. As the exposure time increased beyond 2.5 h, the 3D amplification curves became saturated before 40 cycles of qPCR. I was concluded that the upregulation of CDKN1 A expression caused by MMS treatment was positively correlated with treating time duration in the range of 0.5 to 4.5 h.

To further investigate the effect of MMS treatment on gene expression levels of CDKNI A in single cells, MCF-7 cells were treated with different doses of MMS and then on-chip RT-qPCR was performed. First, different doses of MMS (from 30 to 150 μg/mL) were mixed with cells (˜1×10⁶ cells in a 10 cm dish) in five cultures for 2.5 h at 37° C. in an incubator. After treating, cells were introduced to the arrayed microfluidic device for single-cell processing and RT-qPCR following the above described protocol.

In the five separate analysis units, with the dosage increasing from 30 to 150 μg/ml, the mean Cq value decreased from 33.5 to 29.3 while the standard deviation of this Cq decreased from 0.5 to 0.2 (FIG. 30D (each test was repeated three times and error bars represent standard deviations)). In addition, by plotting the mean qPCR data in a three-dimensional bar graph, it can be found that after 40 PCR cycles the amplification of samples which were treated by MMS with doses from 60 to 150 μg/ml became saturated. While, for the 30 μg/ml MMS-treated sample, the amplification yield at the end of 40-cycle PCR was lower than the other four cases. Furthermore, the inter-unit Cq differences for CDKN1 A were found to be 0.4, 0.7, 1.3 and 0.7, indicating that the intracellular DNA damage was greatest when MMS dosage increased from 90 to 120 μg/ml. As the cells were from the same generation of MCF-7 cell line, it was concluded that the upregulation of CDKNI A expression caused by MMS treatment was positively correlated with MMS dosage when it ranged from 30 to 150 μg/mL.

The results demonstrated that the presented microfluidic array is capable of detecting alterations in transcript levels of single cells and can perform parallelized single-cell analysis.

The foregoing merely illustrates the principles of the disclosed subject matter. Various modifications and alterations to the described embodiments will be apparent to those skilled in the art in view of the teachings herein. Features of existing methods can be integrated into the methods of the exemplary embodiments of the disclosed subject matter or a similar method. It will thus be appreciated that those skilled in the art will be able to devise numerous methods which, although not explicitly shown or described herein, embody the principles of the disclosed subject matter and are thus within its spirit and scope.

Various publications, patents and patent application are cited herein, the contents of which are hereby incorporated by reference in their entireties.

1. A microfluidic device for single cell gene expression analysis comprising:
a cell inlet configured to receive a fluid containing a plurality of cells; and
one or more analysis units coupled to the cell inlet, wherein each of the one or more analysis units comprise:
a cell trap configured to trap a single cell from the plurality of cells;
a reaction chamber coupled to the cell trap;
one or more magnetic microbeads comprising a primer configured to capture mRNA obtained by lysing the single cell; and
one or more magnets configured to transport the one or more microbeads between the cell trap and the reaction chamber.

2. The microfluidic device of claim 1, wherein the cell trap comprises a flow constriction formed by a narrowing in a microchannel between a first microvalve and a second microvalve.

3. The microfluidic device of claim 2, further comprising a cell trapping outlet allowing the fluid to flow through the trap and a cell washing outlet for purging the device of excess cells.

4. The microfluidic device of claim 2, wherein the flow constriction is neck-shaped.

5. The microfluidic device of claim 2, wherein the first and the second microvalves comprise pressurized control valves.

6. The microfluidic device of claim 5, wherein the pressurized valves comprise an elastomeric material.

7. The microfluidic device of claim 1, wherein the reaction chamber further comprises an evaporation barrier embedded in a top portion thereof.

8. The microfluidic device of claim 7, wherein the evaporation barrier comprises an embedded polyethylene or polycarbonate layer.

9. The microfluidic device of claim 1, further comprising a heater disposed underneath the reaction chamber.

10. The microfluidic device of claim 9, wherein the heater comprises a resistive heater.

11. The microfluidic device of claim 1, further comprising a temperature sensor configured to measure a temperature of the reaction chamber.

12. The microfluidic device of claim 1, wherein the device comprises polydimethylsiloxane.

13. The microfluidic device of claim 1, wherein the primer comprises oligo(dT)₂₅.

14. The microfluidic device of claim 1, further comprising a processor coupled to a memory configured for operations comprising:
trapping a single cell in the cell trap;
removing excess cells from the device;
lysing the single cell with a lysis buffer;
capturing mRNA obtained by lysing the single cell with the one or more magnetic microbeads;
transferring the one or more magnetic microbeads to the reaction chamber; and
conducting gene expression analysis of the captured mRNA in the reaction chamber.

15. The microfluidic device of claim 1, comprising two or more analysis units comprising two or more microvalves each disposed upstream of the two or more analysis units, wherein the two or more microvalves are configured to allow the fluid to flow from the cell inlet to any one of the two or more analysis units and isolate the rest of the two or more analysis units from the cell inlet.

16. A method for single cell gene expression analysis comprising:
(a) providing a microfluidic device comprising: a cell inlet and one or more analysis units coupled to the cell inlet, wherein each of the one or more analysis units comprise a cell trap, a reaction chamber coupled to the cell trap, one or more magnetic microbeads comprising at least one primer configured to capture cell mRNA, and one or more magnets;
(b) optionally isolating one or more analysis units from the cell inlet;
(c) trapping a single cell in the cell trap of at least one analysis unit;
(d) removing excess cells from the device;
(e) lysing the single cell with a lysis buffer;
(f) capturing mRNA obtained by lysing the single cell with the one or more magnetic microbeads comprising at least one primer;
(g) transferring the one or more magnetic microbeads to the reaction chamber of the selected analysis unit for gene expression analysis reactions using the one or more magnets.

17. The method of claim 16, wherein only one analysis unit is used at a time and steps (b)-(f) are repeated to prepare another of the one or more analysis units for the gene expression analysis.

18. The method of claim 16, wherein one or more of the one or more analysis units serve as a no-template control.

19. The method of claim 16, further comprising thermal treatments for conducting the gene expression analysis reactions.

20. The method of claim 19, wherein the gene expression analysis reactions comprise RT and qPCR.

21. The method of claim 20, wherein the gene expression analysis comprises hydrolysis probe and primer sets.

22. The method of claim 20, wherein the primer is oligo(dT)₂₅.

23. The method of claim 22, wherein products of the gene expression analysis reactions are analyzed with a fluorescence microscope.
